(19) United States
(12) Patent Application Publication
(10) Pub. No.: US 2010/0112017 A1
(43) Pub. Date: May 6, 2010

(54) Composition For The Preparation of Cosmetics, Cosmetic, and Method For the Preparation Of Water-Containing Cosmetics

(76) Inventors: Yasue Mizutani, Chiba (JP); Hidetoshi Kondo, Chiba (JP); Tomoko Oto, Kanagawa (JP)

Correspondence Address: HOWARD & HOWARD ATTORNEYS PLLC, 450 West Fourth Street, Royal Oak, MI, 48067, US

(21) Appl. No.: 12/441,268

(22) PCT Filed: Sep. 14, 2007
(86) PCT No.: PCT/JP2007/068496

§ 371 Date: Dec. 10, 2009

(30) Foreign Application Priority Data
Sep. 14, 2006 (JP) JP2006-250121
Sep. 14, 2007 (JP) PCT/JP2007/068496

(51) Int. Cl. A61K 8/89; A61K 8/06

(52) U.S. Cl. 424/401; 424/78.03

(57) ABSTRACT

Composition for the preparation of cosmetics comprising a mixture of (a) a polyoxyalkylene-modified diorganopolysiloxane of the formula: (A(R¹)₂SiO{(R¹)₂SiO}ₘ{(R¹)(R²)SiO}ₙSi(R)₂A {where R¹ is monovalent hydrocarbon group (except for R²); R² is polyoxyalkylene group of the formula: —R³O—(C₂H₄O)ₐ(C₃H₆O)_{b}R⁴) [where R³ is alkylenei R⁴ is H, alkyl, or alkoxy], 1=<a=<50; 0=<b=<50; and 10=<(a+b)=<100]; “A” is OH, R¹ or R²; 100=<m=<500; 0=<n=<40; when n=0, at least one “A” is R²}; (b) surfactant, and (c) oil; Composition therefor further comprising (d) biologically allowable hydrophilic medium; Composition therefor further comprising water,’ Cosmetic or OAV type emulsion cosmetic containing the aforementioned composition! and Method for preparing water-containing cosmetics by mixing said composition with water.

### TECHNICAL FIELD

The present invention relates to a composition for the preparation of cosmetics comprising a specifically structured polyoxyalkylene-modified diorganopolysiloxane, a surfactant and an oil, a composition for the preparation of cosmetics further comprising a biologically allowable hydrophilic medium, and a composition for the preparation of cosmetics further comprising water. The present invention also relates to a cosmetic, in particular, to a water-in-oil-type emulsion cosmetic, which contains the aforementioned composition for the preparation of cosmetics and is characterized by excellent temporal stability, sensory feel, moisture retention, external appearance of the product, etc. The present invention further relates to a simple method for the preparation of a water-containing cosmetic, which is characterized by excellent temporal stability, sensory feel, moisture retention, external appearance of the product, etc., by mixing the aforementioned composition for the preparation of cosmetics with water.

### BACKGROUND ART

Due to such properties as excellent spreadability, ability of imparting a refreshed feel, lubricity, water-repelling properties, safety, etc., silicone oils find wide applications in the manufacture of cosmetic products. However, silicone oils have properties significantly different from other oils, and in order to prepare a cosmetic with a silicone oil easily dissolved or dispersed in water or in an aqueous solution of ethanol, it is necessary to optimize the composition and emulsification conditions by using specific large-scale emulsification equipment. However, it has not always happened that the composition prepared for optimized emulsification conditions provides a cosmetic with optimized properties, and a lot of labor and cost must be spent, especially for cosmetics with a silicone oil, in order to optimize compounding and emulsification conditions for obtaining a cosmetic characterized by excellent temporal stability, sensory feel, moisture retention, and external appearance of the product, etc.

For example, Japanese Unexamined Patent Application Publication (hereinafter referred to as “Kokai”) 2000-313808 (JP 2000-313808 A) discloses a soluble composition consisting of a silicone oil, a high-molecular polyoxyalkylene-modified diorganopolysiloxane, ethanol, and water. The aforementioned soluble composition makes it possible to emulsify an oil such as a silicone oil and hydrocarbon oil, in a large amount of water by means of the high-molecular polyether-modified silicone. However, in order to prepare an emulsion which is transparent, has uniform appearance and excellent temporal stability, as well as particle diameters smaller than 1 μm (1,000 nm), it is necessary to optimize emulsification conditions in the preparation thereof, and therefore such an emulsion cannot be easily obtained.

Similarly, Kokai H07-291825 (JP H07-291825 A) proposes an oil-in-water-type cosmetic composed of a silicone oil, an oiling agent for dissolving the silicone oil, a polyether-modified silicone, a surfactant, and water. However, for dissolving the silicone oil, the aforementioned oil-in-water-type cosmetic requires that the oiling agent for the dissolving silicone oil be used in large quantities. In addition, it is difficult to decide an optimized emulsification condition during the manufacturing process.

Kokai 1109-175933 (JP H09-175933 A) proposes a transparent emulsion having diameter of particles less than 100 nm and composed of a polyether-modified silicone, a silicone oil, and an ionic amphipathic lipid. However, the preparation thereof requires the use of special emulsification equipment such as a high-pressure emulsifier represented by a high-pressure homogenizer, and such an emulsion cannot be easily prepared.

### SUMMARY OF THE INVENTION

It is an object of the present invention to provide a composition for the preparation of cosmetics, which is possible to prepare a stable oil-in-water type emulsion without the use of any special emulsifier but rather with the use of a simple stirrer or mixer and practically does not need to find any optimized emulsification condition. It is another object to provide a composition for the preparation of cosmetics, which is possible to emulsify in water and to prepare a stable oil-in-water type emulsion having emulsion particles of small-diameter without the use of any special emulsifier but rather with the use of a simple stirrer or a mixer and practically does not need to find any optimized emulsification condition.

It is another object to provide a composition for the preparation of cosmetics, which is possible to emulsify in water and to prepare an oil-in-wate type emulsion having excellent temporal stability and emulsion particles of microscopic diameter without the use of any special emulsifier but rather with the use of a simple stirrer or mixer and practically does not need to find any optimized emulsification condition.

A further object of the present invention is to provide a cosmetic characterized by excellent temporal stability, good sensory feel, good moisture retention, attractive external appearance of the product, etc., in particular, a stable oil-in-water emulsion cosmetic having emulsion particles of microscopic diameter. It is still another object to provide a simple method for manufacturing water-containing cosmetics that is characterized by excellent temporal stability, sensory feel, moisture retention, external appearance of the product, etc.

Based on the results of profound studies, the inventors herein have found that the above objects can be achieved by:

(1) a composition for the preparation of cosmetics comprising a mixture of (a) a polyoxyalkylene-modified diorganopolysiloxane represented by the following average structural formula (1):

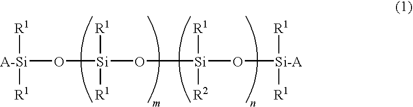

{where R¹ is a monovalent hydrocarbon group or substituted monovalent hydrocarbon group (except for such groups designated by R²); R² is a polyoxyalkylene group of the following general formula (2): —R³—O—(C₂H₄O)ₐ(C₃H₆O)_{b}R⁴ [where R³ is an alkylene group with 2 to 30 carbon atoms; R⁴ is a group selected from hydrogen atom, an alkyl group with 1 to 30 carbon atoms, or an organic group of the following formula: —(OC)—R⁵ (where R⁵ is an alkyl group with 1 to 30 carbon atoms), “a” and “b” are numbers that satisfy the following conditions: 1≧a≧50; 0≧b≧50; and 10≧(a+b)≧100]; and “A” may be the same or different and is selected from hydroxyl group, and R¹ and R²;
“m” and “n” satisfy the following conditions: 100≧m≧500; 0≧n≧40; but when n=0, at least one “A” is R²)};
(b) a surfactant of one or more types (except for surfactants corresponding to component (a)); and
(c) an oil of one or more types;
(2) a composition for the preparation of cosmetics which is a mixture of aforementioned components (a), (b), and (c) with (d) a biologically allowable hydrophilic medium of at least one type; and
(3) a composition for the preparation of cosmetics which is a mixture of aforementioned components (a), (b), (c), and (d) a biologically allowable hydrophilic medium of at least one type with (e) water.
Thus the inventors arrived at the present invention.

The inventors found to be able to solve the problems of the prior art by a composition for the preparation of cosmetics which has an appropriate ratio of components (a), (b), and (c); an appropriate ratio of components (a), (b), (c), and (d), and an appropriate amount of component (d); or an appropriate ratio of components (a), (b), (c), and (d), and appropriate amounts of components (d) and (e), and came to the present invention.

The inventors found to be able to solve the problems of the prior art by a cosmetic that contains the aforementioned composition and is characterized by excellent temporal stability, sensory feel, moisture retention, external appearance of the product, etc., and came to the present invention.

Finally, the inventors found to be able to simply produce water-containing cosmetics characterized by excellent temporal stability, sensory feel, moisture retention, external appearance of the product, etc. by mixing the aforementioned composition for the preparation of cosmetics with water, and came to the present invention.

### BEST MODE FOR CARRYING OUT THE INVENTION

More specifically, the present invention relates to the following:

[1] A composition for the preparation of cosmetics comprising a mixture of (a) a polyoxyalkylene-modified diorganopolysiloxane represented by the following average structural formula (1);

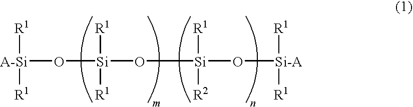

{where R¹ is a monovalent hydrocarbon group or substituted monovalent hydrocarbon group (except for groups corresponding to R²); R² is a polyoxyalkylene group of the following general formula (2): —R³—O—(C₂H₄O)ₐ(C₃H₆O)_{b}R⁴ [where R³ is an alkylene group with 2 to 30 carbon atoms; R⁴ is a group selected from hydrogen atom, an alkyl group with 1 to 30 carbon atoms, or an organic group of the following formula: —(OC)—R⁵ (where R⁵ is an alkyl group with 1 to 30 carbon atoms), “a” and “b” are numbers that satisfy the following conditions: 1≧a≧50; 0≧b≧50; and 10≧(a+b) 100]; and “A” may be the same or different and is selected from hydroxyl group, R¹ and R²; “m” and “n” satisfy the following conditions: 100≧m≧500; 0≧n≧40; but when n=0, at least one “A” is R²)};

(b) a surfactant of one or more types (except for surfactants corresponding to component (a)); and

(c) an oil of one or more types;

[1-1] The composition for the preparation of cosmetics according to Item [1], wherein the composition for the preparation of cosmetics is a cosmetic raw material, i.e., raw material for cosmetics, cosmetic products or cosmetic compositions, or ingredient to be contained in various cosmetics, cosmetic products or cosmetic compositions.
[2] The composition for the preparation of cosmetics according to Item [1], wherein component (a) is present in an amount of 0.85 to 680 parts by mass and component (b) is present in an amount of 1.4 to 1120 parts by mass per 100 parts by mass of component (c);
[3] The composition for the preparation of cosmetics according to Item [2], wherein component (a) is present in an amount of 1.0 to 340 parts by mass and component (b) is present in an amount of 1.5 to 560 parts by mass per 100 parts by mass of component (c);
[4] The composition for the preparation of cosmetics that comprises a mixture of component (a) described in Item [1], component (b) described in Item [1], component (c) described in Item [1], and (d) a biologically allowable hydrophilic medium of one or more types;
[5] The composition for the preparation of cosmetics according to Item [4], wherein component (d) is present in an amount of 20.0 to 98.4% of the total mass of the composition;
[6] The composition for the preparation of cosmetics according to Item [5], wherein component (d) is present in an amount of 40.0 to 98.4% of the total mass of the composition;
[7] The composition for the preparation of cosmetics according to Items [5] or [6], wherein component (a) is present in an amount of 0.9 to 90 parts by mass, component (b) is present in an amount of 0.6 to 120 parts by mass, and component (c) is present in an amount of 1.1 to 150 parts by mass per 100 parts by mass of component (d);
[8] The composition for the preparation of cosmetics according to Item [7], wherein component (a) is present in an amount of 4.0 to 300 parts by mass, and component (b) is present in an amount of 5 to 480 parts by mass per 100 parts by mass of component (c);
[9] The composition for the preparation of cosmetics that comprises a mixture of component (a) described in Item [1], component [b] described in Item [1], component [c] described in Item DJ, component (d) described in Item [4], and (e) water;
[10] The composition for the preparation of cosmetics according to Item [9] wherein component (a) is present in an amount of 0.9 to 90 parts by mass, component (b) is present in an amount of 0.6 to 120 parts by mass, and component (c) is present in an amount of 1.1 to 150 parts by mass per 100 parts by mass of component (d), and wherein component (d) is present in an amount of 20.0 to 98.4% of the total mass of the composition, and component (e) is present in the amount not exceeding the content of component (d) and not exceeding 40.0% of the total mass of the composition for the preparation of cosmetics;
[11] The composition for the preparation of cosmetics according to any of Items [1] to [10], wherein the following condition is observed in the average structural formula (1) of component (a): 5 (m/n) δ 50;
[12] The composition for the preparation of cosmetics according to any of Items [1] to [10], wherein the viscosity of a 50 wt. % dipropyleneglycol solution of component (a) at 25° C. ranges from 1,000 mPa·s to 60,000 mPa·s;
[12-1] The composition for the preparation of cosmetics according to Item [11], wherein the viscosity of a 50 wt. % dipropyleneglycol solution of component (a) at 25° C. ranges from 1,000 mPa·s to 60,000 mPa·s;
[13] The composition for the preparation of cosmetics according to any of Items [1] to [10], wherein component (b) is composed of at least one (b1) an ionic surfactant and at least one (b2) a non-ionic surfactant;
[13-1] The composition for the preparation of cosmetics according to Items [11], [12], or [12-1], wherein component (b) is composed of at least one (bp an ionic surfactant and at least one (b2) a nonionic surfactant;
[14] The composition for the preparation of cosmetics according to Items [13] or [13-1], wherein component (b1) is an anionic surfactant or a phospholipid;
[15] The composition for the preparation of cosmetics according to Item [14], wherein the anionic surfactant is selected from the group consisting of a polyoxyalkylene alkyl ether phosphoric acid, alkali-metal salt thereof, N-fatty acid acylamino acid, alkali-metal salt of polyoxyalkylene sulfosuccinic acid, and alkali-metal salt of sulfosuccinic acid ester of a polyoxyalkylene-modified dimethylpolysiloxane;
[16] The composition for the preparation of cosmetics according to Item [13], wherein component (b2) is a non-ionic surfactant of at least one type selected from the group consisting of a polyoxyalkylene-modified diorganopolytsiloxane (wherein the degree of polymerization in the diorganopolysiloxane portion is lower than that of the diorganopolytsiloxane portion of component (a), and HLB is in the range of 3 to 10), polyoxyalkylene alkyl ether, sorbitane fatty acid ester, polyoxyalkylenesorbitane fatty acid ester, polyoxyethylene hardened castor oil, and polyoxyalkylene fatty acid ester;
[17] The composition for the preparation of cosmetics according to Item [16], wherein the polyorganoalkylene-modified diorganopolysiloxane of component (b2) is represented by the following average structural formula (3):

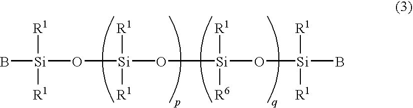

{where R¹ is a monovalent hydrocarbon group or substituted monovalent hydrocarbon group (except for groups corresponding to R⁶); R⁶ is a polyoxyalkylene group of the following general formula (4): —R⁷—O—(C₂H₄O)_{d} (C₃H₆O)ₑ R⁸ [where R⁷ is an alkylene group with 2 to 8 carbon atoms; R⁸ is a group selected from a hydrogen atom, alkyl group with 1 to 12 carbon atoms, and organic group of the following formula: —(OC)—R⁹ (where R⁹ is an alkyl group with 1 to 12 carbon atoms), “d” and “e” are numbers that satisfy the following conditions: 1≦d≦20; 0≦e≦20; and 5≦(d+e)≦40]; groups designated by “B” may be the same or different and are selected from hydroxyl group, R¹ and R⁶; “p” and “q” satisfy the following conditions: 0≦p≦90; 0≦q≦10; but when q=0, at least one “B” is R⁶};
[18] The composition for the preparation of cosmetics according to any of Items [1] to [10], wherein component (c) is a higher fatty acid alkyl ester, hydrocarbon oil, or a hydrophobic silicone oil having a viscosity of 0.65 mPa·s to 100,000 mPa·s at 25° C.;
[18-1] The composition for the preparation of cosmetics according to any of Items [11], [12], or [12-1], wherein component (c) is a higher fatty acid alkyl ester, hydrocarbon oil, or a hydrophobic silicone oil having a viscosity of 0.65 mPa·s to 100,000 mPa·s at 25° C.;
[19] The composition for the preparation of cosmetics according to any of Items [4] to [10], wherein component (d) is a hydrophilic alcohol that has in one molecule at least one hydroxyl group and that is liquid at room temperature;
[19-1] The composition for the preparation of cosmetics according to any of Items [11], [12], or [12-1], wherein component (d) is a hydrophilic alcohol that has in one molecule at least one hydroxyl group and that is liquid at room temperature;
[20] The composition for the preparation of cosmetics according to Items [19] or [19-1], wherein the hydrophilic alcohol is a monovalent or polyvalent alcohol having 2 to 10 carbon atoms;
[21] The composition for the preparation of cosmetics according to Item [20], wherein the monovalent or polyvalent alcohol having 2 to 10 carbon atoms is selected from the group consisting of ethanol, isopropyl alcohol, and dipropyleneglycol;
[22] The composition for the preparation of cosmetics according to any of Items [1] to [21], wherein the composition for the preparation of cosmetics is a composition for the preparation of oil-in-water type emulsion cosmetics;
[23] The composition for the preparation of cosmetics according to any of Items [1] to [3], wherein the composition for the preparation of cosmetics is a composition for the preparation of oil-in-water type emulsion cosmetics with an average particle size, measured by the laser diffraction/dispersion method for emulsion particles, of less than 10.0 μm (10,000 nm);
[24] The composition for the preparation of cosmetics according to any of Items [4] to [10], wherein the composition for the preparation of cosmetics is a composition for the preparation of oil-in-water type emulsion type cosmetics with an average particle size, measured by a laser diffraction/dispersion method for emulsion particles, of less than 0.20 μm (200 nm);
[24-1] The composition for the preparation of cosmetics according to any of Items [11], [12], or [12-1], wherein the composition for the preparation of cosmetics is a composition for the preparation of oil-in-water type emulsion cosmetics with an average particle size, measured by a laser diffraction/dispersion method for emulsion particles, of less than 0.20 μm (200 nm);
[25] The composition for the preparation of cosmetics according to Item [4], wherein the composition for the preparation of cosmetics is a composition for the preparation of oil-in-water type emulsion cosmetics characterized by the fact that component (d) is present in the amount of 55.0 to 98.4% of the total mass of the composition, and component (a) is present in the amount of 0.9 to 30 parts by mass, component (b) is present in the amount of 0.6 to 40 parts by mass, and component (c) is present in the amount of 1.1 to 50 parts by mass per 100 parts by mass of aforementioned component (d);
component (d) is ethanol alone or a mixture of ethanol with a biologically allowable hydrophilic medium other than ethanol (however, the mass ratio of the aforementioned components in the mixture should be not less than 6/4);
component (b) consists of (b1) an ionic surfactant of one or more types and (b2) a nonionic surfactant of one or more types; and
component (c) is a hydrophobic silicone oil having a viscosity of 0.65 mPa·s to 100,000 mPa·s at 25° C., and
an average particle size, measured by a laser diffraction/dispersion method for emulsion particles, is less than 0.10 μm (100 nm);
[26] The composition for the preparation of cosmetics according to any of Items [1] to [25], wherein the composition for the preparation of cosmetics is a composition for the preparation of skin cosmetics;
[27] A cosmetic that contains the composition for the preparation of cosmetics described in any of Items [1] to [25];
[28] An oil-in-water type emulsion cosmetic that contains the composition for the preparation of cosmetics described in any of Items [22], [23], [24], and [25].
[29] The cosmetic according to Items [27] or [28], wherein the cosmetic is a skin cosmetic;
[30] A method for the preparation of water-containing cosmetics comprising mixing the composition for the preparation of cosmetics described in any of Items [1] to [25] with water.

The inventive composition for the preparation of cosmetics that comprises a mixture of components (a), (b), and (c) can be emulsified with water without the use of any special emulsifier but rather with the use of a simple stirrer or a mixer to form a stable oil-in-water type emulsion with microscopic particles, and practically does not require any preparatory optimization of emulsification conditions. Normally, the inventive composition for the preparation of cosmetics that comprises a mixture of components (a), (b), (c) and (d) is transparent, can be emulsified with water without the use of any special emulsifier but rather with the use of a simple stirrer or a mixer, to form a stable oil-in-water type emulsion with microscopic particles, and practically does not require any preparatory optimization of emulsification conditions.

The inventive composition for the preparation of cosmetics that comprises a mixture of components (a), (b), (c), (d) and (e) can be emulsified with water without the use of any special emulsifier but rather with the use of a simple stirrer or a mixer to form a stable oil-in-water type emulsion with microscopic particles, and practically does not require any preparatory optimization of emulsification conditions. The inventive composition has low flammability under industrial conditions and therefore is safe in handling. The cosmetic of the present invention is a cosmetic, in particular oil-in-water type emulsion cosmetic having microscopic size of particles, characterized by excellent temporal stability, sensory feel, moisture retention, external appearance of the product, etc.

The inventive method for the preparation of water-containing cosmetics makes it possible to simply prepare water-containing cosmetics that are characterized by excellent temporal stability, sensory feel, moisture retention, external appearance of the product, etc.

The composition for the preparation of cosmetics of the present invention is characterized by comprising a mixture of (a) a polyoxyalkylene-modified diorganopolysiloxane represented by the following average structural formula (1);

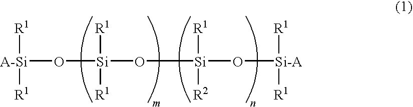

{where R¹ is a monovalent hydrocarbon group or substituted monovalent hydrocarbon group (except for groups corresponding to R²); R² is a polyoxyalkylene group of the following general formula (2): —R³—O—(C₂H₄O)ₐ(C₃H₆O)_{b}R⁴ [where R³ is an alkylene group with 2 to 30 carbon atoms; R⁴ is a group selected from hydrogen atom, an alkyl group with 1 to 30 carbon atoms, or an organic group of the following formula: —(OC)—R⁵ (where R⁵ is an alkyl group with 1 to 30 carbon atoms), “a” and “b” are numbers that satisfy the following conditions: 1≦a≦50; 0≦b≦50; and 10≦(a+b)≦100]; and “A” may be the same or different and is selected from hydroxyl group, R¹ and R²; “m” and “n” satisfy the following conditions: 100≦m≦500; 0≦n≦40; but when n=0, at least one “A” is R²)};

(b) a surfactant of one or more types (except for surfactants corresponding to component (a)); and

(c) an oil of one or more types.

Since the polyoxyalkylene-modified diorganopolysiloxane, that is component (a), contains 100 to 500 (R¹)₂SiO units, which are diorganopolysiloxane units with hydrophobic and lipophilic properties, and contains groups R², which are hydrophilic groups, this component can be classified as a surfactant. Although there are no special restrictions with regard to HLB of this component, it is recommended that HLB be in the range of 5 to 13.

In the aforementioned composition for preparation of cosmetics, the polyoxyalkylene-modified diorganopolysiloxane that is component (a) is well compatible with one or more surfactants that is component (b) and one or more oils that is component (c). Furthermore, when an oil-in-water type emulsion cosmetic is prepared by mixing the aforementioned composition with water and whereby emulsifying component (c) in water, component (a) together with component (b) accelerates the process of emulsification in water, facilitates emulsification of component (c) without using special equipment, and imparts to the prepared emulsion good temporal stability.

The polyoxyalkylene-modified diorganopolysiloxane, that is component (a), is represented by the following average structural formula (1):

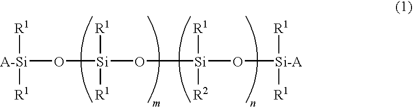

wherein R¹ is an unsubstituted or substituted monovalent hydrocarbon group (except for groups corresponding to R²). The unsubstituted monovalent hydrocarbon group is exemplified by methyl, ethyl, propyl, or similar alkyl groups; phenyl, tolyl, xylyl, or similar aryl groups; or aralkyl groups. The substituted monovalent hydrocarbon group is exemplified by 3,3,3-trifluoropropyl, 3,3,4,4,4-pentafluorobutyl, or similar perfluoroalkyl groups; methoxycarbonylpropyl, ethoxycarbonylpropyl, or similar alkoxycarbonylalkyl groups; acetoxypropyl, propionoxypropyl, or similar alkylcarbonyloxyalkyl groups; ethoxyethyl, or similar alkoxyalkyl groups; 3-aminopropyl, 3-(aminoethyl)aminopropyl, or similar aminoalkyl groups; alkylaminocarbonylalkyl, or alkylcarbonylaminoalkyl groups. The groups corresponding to R² are excluded from the aforementioned hydrocarbon groups. A part of groups represented by R¹ bonded to silicon atoms, especially a part of groups represented by R¹ on the molecular terminals, may be replaced by an alkoxy group or alkoxy groups, such as methoxy, ethoxy, or propoxy groups.

Among the above, R¹ is preferably the unsubstituted monovalent hydrocarbon group, preferably the alkyl group, and, in particular, methyl group.

Among the structures represented by the average structural formula (1), a diorganopolysiloxane of the average structural formula (1-1) shown below is preferable from the viewpoint of properties and ease of synthesis.

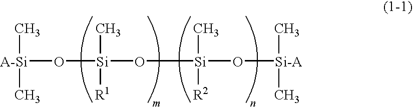

It is the best that all R¹s in the above formula are methyl groups. However, in addition to methyl groups, a part of R¹s may be unsubstituted monovalent hydrocarbon groups (e.g., alkyl groups other than methyl groups or phenyl groups) or substituted monovalent hydrocarbon groups (such as perfluoroalkyl, alkoxycarbonylalkyl, alkylcalbonyloxyalkyl, alkoxyalkyl, or similar hydrophobic organic groups).

In aforementioned average structural formulas (1) and (1-1), R² is a polyoxyalkylene group represented by the following formula (2): —R³—O—(C₂H₄O)ₐ (C₃H₆O)_{b}R⁴ [where R³ is an alkylene group with 2 to 30 carbon atoms; R⁴ is a group selected from hydrogen atom, an alkyl group with 1 to 30 carbon atoms, or an organic group of the following formula: —(OC)—R⁵ (where R⁵ is an alkyl group with 1 to 30 carbon atoms)]. From the viewpoint of required properties, it is recommended that R⁴ is hydrogen atom, R³ that represents the aforementioned alkylene group should preferably have 2 to 6 carbon atoms, and most preferably, 3 or 4 carbon atoms. The alkyl groups that are represented by R⁴ and R⁵ preferably have 1 to 8 carbon atoms. In the above formula, “a” and “b” should satisfy the following conditions: 1≦a≦50; 0≦b≦50, and 10≦(a+b)≦100, and (a+b) preferably should be in the range of 30 to 50. “A” is the same or different groups selected from hydroxyl group, R¹, and R².

In the above formula, “m” and “n” satisfy the following conditions: 100≦m≦500; 0≦n≦40; but when n=0, at least one “A” is R² but it is more preferable that both “A” are R². It is preferable that “m” ranges from 100 to 400, and “n” ranges from 0 to 20, and the ratio m/n satisfies the following condition: 5≦(m/n)≦50.

If the values of “m” and “n” exceed the recommended upper limit, this will significantly increase viscosity and impair handling properties. If the value of (a+b) is below the recommended lower limit, this will impair emulsification conditions, and if the aforementioned value exceeds the recommended upper limit, this will complicate production. Furthermore, if the value of “m” is below the recommended lower limit, this will impair temporal stability not only of the emulsion but temporal stability of cosmetics as well.

There are no special restrictions with regard to the sequence of siloxane units in the aforementioned average structural formulas (1) and (1-1), and the sequence of these units may be random, blocked, or a combination of both. The sequence of siloxane units in copolymers is random in general. The same is true for —(C₂H₄O)ₐ(C₃H₆O)_{b}—.

Component (a) should have an average molecular weight ranging preferably from 10,000 to 80,000 and more preferably from 20,000 to 60,000. If the molecular weight is below the recommended lower limit, this will impair temporal stability of cosmetics, and if the molecular weight exceeds the recommended upper limit, this will impair the sensory feel of cosmetics.
There are no special restrictions with regard to viscosity of component (a); however, from the viewpoint of preparing cosmetics with improved temporal stability and smoothness of touch, it is recommended that the viscosity of component (a) at 25° C. in a 50-wt. % solution of dipropyleneglycol ranges preferably from 500 to 100,000 mPa·s and more preferably from 1,000 to 60,000 mPa·s.

The polyoxyalkylene-modified diorganopolysiloxanes represented by average structural formulas (1) and (1-1) are exemplified more specifically by a group of diorganopolysiloxanes, the average structural formulas of which are given below. In these formulas, the values of “m”, “n” and “a” are in the numerical ranges given above.

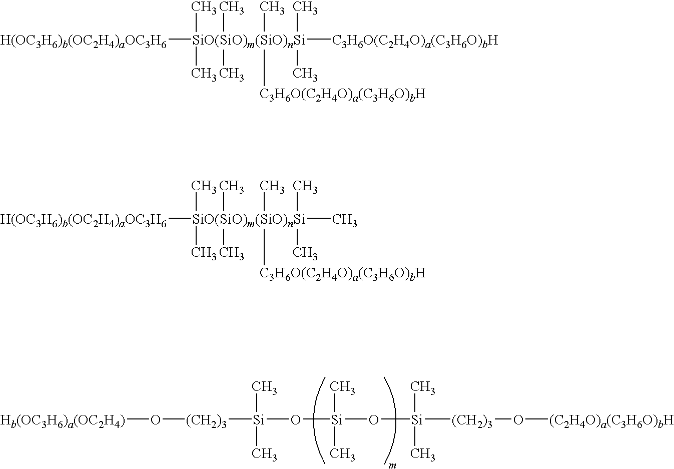

Component (a) may be composed of two or more molecules which are different in siloxane units, degrees of polymerization, silicon-bonded substituents, polyoxyalkylene groups, endblocking groups etc.

There are no special restrictions with regard to the method of manufacturing the high-molecular polyoxyalkylene-modified diorganopolysiloxane of the average structural formula (1). For example, this compound can be produced by causing a hydrosilylation reaction between (A) a linear-chain organopolysiloxane having silicon-bonded hydrogen atoms and represented by average structural formula (5) given below:

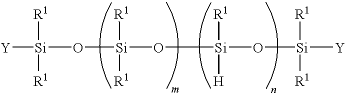

(where R¹ is a monovalent hydrocarbon group or a substituted monovalent hydrocarbon group (except for groups corresponding to R²); Y on the molecular terminals is the same or different groups selected from R¹, hydroxyl group and alkoxy group; and “m” and “n” satisfy the following conditions: 100≦m≦500; 0≦n≦40; however, when n=0, at least one Y on the molecular terminal should be a hydrogen atom) and (B) a polyoxyalkylene compound having an alkenyl group on its molecular terminal and represented by the following general formula (6):
R⁶—O—(C₂H₄O)ₐ(C₃H₆O)_{b}R⁴ [where R⁴ is a group selected from hydrogen atom, an alkyl group with 1 to 30 carbon atoms, and an organic group of the following formula:
—(OC)—R⁵ (where R⁵ is an alkyl group with 1 to 30 carbon atoms); and R⁶ is an alkenyl group having 2 to 30 carbon atoms], the reaction being carried out in the presence of (C) a hydrosilylation catalyst.
The aforementioned linear-chain organopolysiloxane that has silicon-bonded hydrogen atoms can be produced conventionally by equilibrium polymerization.

R¹, a group other than hydrogen atoms bonded to silicon atoms contained in diorganopolysiloxane (A) is the same as R¹ in average structural formula (1), and is exemplified by unsubstituted monovalent hydrocarbon groups such as methyl, ethyl, propyl, butyl, pentyl, heptyl, octyl, or a similar alkyl group; phenyl, tolyl, xylyl, or a similar aryl group; benzyl, phenethyl, or a similar aralkyl group; 3-chloropropyl, 3,3,3-trifluoropropyl, or a similar halogenated alkyl group; an alkoxycarbonylalkyl group; an alkylcarbonyloxyalkyl group, an alkoxyalkyl group, or similar substituted hydrocarbon groups that are free of unsaturated aliphatic bonds. Methoxy, ethoxy, propoxy, or similar alkoxy or hydroxyl groups may bond to the molecular terminals. The unsubstituted monovalent hydrocarbon group, in particular, alkyl group, and, more specifically, methyl group is preferable as R¹.

In (B) polyoxyalkylene that has an alkenyl group on the molecular terminal and is represented by the following formula (6): R⁶—O—(C₂H₄O)ₐ(C₃H₆O)_{b}R⁴, R⁴ is a hydrogen atom, an alkyl group with 1 to 30 carbon atoms, or an organic group of formula —(OC)—R⁵ (where R⁵ is an alkyl group with 1 to 30 carbon atoms); and R⁶ is an alkenyl group with 2 to 30 carbon atoms which is exemplified by a vinyl, allyl, butenyl, pentenyl, hexenyl, heptenyl, octenyl, nonenyl, or decenyl group, of which vinyl, allyl, butenyl, or hexenyl groups are preferable.

(C) a hydrosilylation catalyst is a catalyst for accelerating the hydrosilylation reaction between the silicon-bonded hydrogen atoms of organopolysiloxane (A) and the alkenyl groups of polyoxyalkylene compound (B). Such a catalyst is exemplified by a platinum-type catalyst, a rhodium-type catalyst, or a palladium-type catalyst, of which the platinum-type catalyst is preferable. The platinum-type catalyst, in turn, is exemplified by chloroplatinic acid, alkoxy-modified chloroplatinic acid, an olefin complex of platinum, a ketone complex of platinum, a divinylsiloxanetetramethyldisiloxane complex of platinum, platinum tetrachloride, a fine platinum powder, solid platinum supported on alumina or silica carriers, platinum black, an olefin complex of platinum, a complex of chloroplatinic acid and divinylsiloxanetetramethyldisiloxane, a carbonyl complex of platinum; or methylmethacrylate resin, polycarbonate resin, polystyrene resin, silicone resin, or a similar thermoplastic organic resin powder containing the aforementioned platinum catalyst. Most preferable is chloroplatinic acid. There are no special restrictions with regard to an amount of the hydrosilylation catalyst provided that it is used in an effective amount. However, it is recommended that the aforementioned catalyst be used in an amount such that the content of metallic platinum in component (C), in terms of weight units, be in the range of 0.1 to 1,000 ppm, and, preferably, 0.5 to 100 ppm per total weight of organopolysiloxane (A) and polyoxyalkylene compound (B).

The aforementioned hydrosilylation reaction may be carried out in an organic solvent. Preferable examples of the organic solvent are the following: isopropyl alcohol or a similar alcohol-type solvent; toluene, xylene, or a similar aromatic hydrocarbon-type solvent; dioxane, THF or a similar ethyl-type solvent; an aliphatic hydrocarbon-type solvent; carboxylic acid ester-type solvent, ketone-type solvent, or a chlorinated hydrocarbon-type solvent. When the aforementioned organic solvent is used, it is preferable that it is distilled from the product after completion of the reaction by adding an evaporation-resistant diluent such as dipropyleneglycol and stripping the product under reduced pressure. This is necessary to prevent situations wherein cosmetics, specifically skin cosmetics contain organic solvents other than alcohol-type solvents.

There are no special limitations with regard to the temperature at which the aforementioned hydrosilylation reaction is carried out, but generally the reaction is carried out preferably at a temperature ranging from 50 to 150° C.
On completion of the hydrosilylation reaction, an unpleasant odor can be eliminated by removing allylether groups with the use of an acidic substance or by performing alkylation that can be carried out by hydrogenation.
Tocopherol or BHT can be added to the obtained polyoxyalkylene-modified diorganopolysiloxane as an antioxidant.

One or more surfactants that is component (b) in the composition for the preparation of cosmetics is compatible with the polyoxyalkylene-modified diorganopolysiloxane that is component (a). These surfactants accelerat emulsification of component (c) in water together with component (a) when the aforementioned composition is mixed with water to emulsify component (c) in water. As compared to the composition wherein component (b) is used alone, the use of this component (b) in combination with component (a) improves temporal stability of a cosmetic, especially an oil-in-water type emulsion cosmetic.

There are no special restrictions with regard to the aforementioned surfactant (b) of one or more types provided that it is different from component (a) and is generally used for conventional cosmetics, especially oil-in-water type emulsion cosmetics. Examples of this surfactant include an ionic surfactant, nonionic surfactant, or a combination of both. From the viewpoint of improved temporal stability of the oil-in-water type emulsion cosmetic prepared by mixing the composition for the preparation of cosmetics with water, it is preferable to use (b1) an ionic surfactant of one or more types with (b2) a nonionic surfactant of one or more types.

More specifically, examples of (b1) the ionic surfactant of one or more types include an anionic surfactant, cationic surfactant, or amphoteric surfactant. Examples of the anionic surfactant include saturated or unsaturated fatty acid salts (e.g., sodium laurate, sodium stearate, sodium oleate, and sodium linoleate, etc.), long-chain alkylsulfuric acid salts, alkylbenzene sulfonic acids (e.g., hexylbenzene sulfonic acid, octylbenzene sulfonic acid, and dodecylbenzene sulfonic acid, etc.) and their salts, polyoxyalkylene alkyl ether sulfates, polyoxyalkylene alkenyl ether sulfates, polyoxyethylene alkyl sulfate ester salts, sulfosuccinic acid alkyl ester salts, polyoxyalkylene sulfosuccinic acid salts, polyoxyalkylene sulfosuccinic acid ester salts, alkali-metal salt of sulfosuccinic acid ester of polyoxyalkylene-modified dimethylpolysiloxane, polyoxyalkylene alkyl phenyl ether sulfates, long-chain alkanesulfonic acid salts, long-chain alkylsulfonate, polyoxyethylene alkyl phenyl ethyl sulfates, polyoxyalkylene alkyl ether acetates, long-chain alkylphosphates, polyoxyalkylene alkyl ether phosphates, acylglutamic acid salts, a acylsulfonic acid salts, long-chain alkylsulfonic acid salts, alkylallyl sulfonic acid salts, long-chain α-olefin sulfonates, alkylnaphthalenesulfonic acid salts, long-chain alkane sulfonic acid salts, long-chain alkyl or alkenylsulfonic acid salts, long-chain alkylamidesulfonic acid salts, long-chain alkyl or alkenylphosphoric acid salts, alkylamide phosphates, alkyloylalkyltaurine salts, N-acylaminoacid salts, sulfosuccinic acid salts, alkylalkyl ether carboxylic acid salts, amide ether carboxylates, α-sulfo fatty acid ester salts, alanine derivatives, glycine derivatives, or arginine derivatives. Examples of the aforementioned include sodium salts, potassium salts, or similar alkali metal salts; triethanolamine salts or similar alkanolamine salts, as well as ammonium salts, but sodium salts are preferable,

Examples of cationic surfactants include alkyltrimethyl ammonium chloride, stearyltrimethyl ammonium chloride, lauryltrimethyl ammonium chloride, cetyltrimethyl ammonium chloride, beef tallow alkyltrimethyl ammonium chloride, behenyltrimethyl ammonium chloride, octyltrimethyl ammonium hydroxide, dodecyltrimethyl ammonium hydroxide, stearyltrimethyl ammonium bromide, behenyltrimethyl ammonium bromide, distearyldimethyl ammonium chloride, dicocoyldimethyl ammonium chloride, dioctyldimethyl ammonium chloride, di-(POE) oleylmethyl ammonium chloride (2EO), benzalkonium chloride, alkylbenzalkonium chloride, alkyldimethylbenzalkonium chloride, benzethonium chloride, stearyldimethylbenzyl ammonium chloride, lanolin-derived quaternary ammonium salts, stearic acid diethylaminoethylamide, stearic acid dimethylaminopropylamide, behenic acid amide propyldimethyl hydroxypropyl ammonium chloride, stearoycol aminoformylmethylpyridinium chloride, cetylpyridinium chloride, tall oil alkylbenzylhydroxyethyl imidazolinium chloride, or benzyl ammonium salts.

Examples of the amphoteric surfactants include phospholipids, such as lecithins, phosphatidyl ethanolamine, phosphatidic acid, phosphatidyl inositol, phosphatidyl serine, phosphatidyl choline, phosphatidyl glycerol, sphingomyelin, cardiolipin, or hydrogenates of the above compounds. Most preferable are hydrogenated soybean lecithin, egg yoke lecithin, rape lecithin, or a similar hydrogenated natural lecithin.

The nonionic surfactants (b2) of one or more types (except for those corresponding to component (a)) is exemplified by polyoxyalkylene ethers, polyoxyalkylene alkyl ethers, polyoxyalkylene fatty acid esters, polyoxyalkylene fatty acid diesters, polyoxyalkylene resinic acid esters, polyoxyalkylene (hydrogenated) castor oils, polyoxyalkylene alkyl phenols, polyoxyalkylene alkyl phenyl ethers, polyoxyalkylene phenyl phenyl ethers, polyoxyalkylene alkyl esters, olyoxyalkylene alkyl esters, sorbitan fatty acid esters, polyoxyalkylene sorbitan alkyl esters, polyoxyalkylene sorbitan fatty acid esters, polyoxyalkylene sorbitol fatty acid esters, polyoxyalkylene glycerin fatty acid esters, polyglycerol alkyl ethers, polyglycerol fatty acid esters, sucrose fatty acid esters, fatty acid alkanolamides, alkylglucosides, polyoxyalkylene fatty acid bisphenyl ethers, polypropylene glycols, polyether-modified silicones, i.e., polyoxyalkylene-modified diorganopolysiloxanes, (except for those corresponding to component (a)), polyglyceryl-modified silicones, glyceryl-modified silicones, saccharide-modified silicones, perfluoropolyether-type surfactants, polyoxyethylene-polyoxypropylene block copolymers, and alkyl polyoxyethylene-polyoxypropylene block copolymer ethers.

From the viewpoint of improved stability and feel of touch of cosmetics, component (b1) is preferably an anionic surfactant, such as polyoxyalkylene alkylether phosphoric acid, an alkali metal salt thereof (especially, a sodium salt or a potassium salt), or polyoxyalkylene sulfosuccinic acid salt. In particular, the use of an alkali metal salt of polyoxyalkylene sulfosuccinic acid (especially, a sodium salt or a potassium salt), and a sulfosuccinic acid ester alkali metal salt of polyoxyalkylene-modified dimethylpolysiloxane makes it possible to obtain compositions for the preparation of cosmetics with reduced unpleasant odor and reduced irritation of skin, and this contributes to improvement in the properties of the cosmetics. Furthermore, from the viewpoint of improved stability and feel of touch, the nonionic surfactant is preferably polyoxyalkylene alkylethers or polyoxyalkylene fatty acid esters. When it is necessary to improve temporal stability of cosmetics, especially, oil-in-water type emulsion type cosmetics, it is recommended to use polyether-modified silicones, i.e., polyoxyalkylene-modified diorganopolysiloxane (except for those corresponding to component (a)), polyglycerol-modified silicones, glycerol-modified silicone, or saccharide-modified silicones. Since these modified silicones have the same diorganopolysiloxane skeletons as component (a), they can be used in conjunction with component (a), and this, in turn, improves temporal stability of cosmetics, especially, oil-in-water type emulsion cosmetics.

From the viewpoint of temporal stability of cosmetics, especially of oil-in-water type emulsion cosmetics, the nonionic surfactant that is component (b2) is preferably polyoxyalkylene-modified diorganopolysiloxane of average structural formula (3) given below:

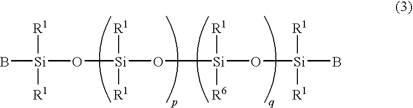

{where R¹ is a monovalent hydrocarbon group or substituted monovalent hydrocarbon group (except for groups corresponding to R⁶); R⁶ is a polyoxyalkylene group of the following general formula (4):
—R⁷—O—(C₂H₄O)_{d}(C₃H₆O)ₑR⁸ [where R⁷ is an alkylene group with 2 to 8 carbon atoms; R⁸ is a group selected from hydrogen atom, an alkyl group with 1 to 12 carbon atoms, and an organic group of the following formula: —(OC)—R⁹ (where R⁹ is an alkyl group with 1 to 12 carbon atoms); “d” and “e” are numbers that satisfy the following conditions: 1≦d≦20; 0≦e≦20; 5≦(d+e)≦40];
“B” is a group that may be the same or different and is selected from hydroxyl group, R¹ and R⁶; “p” and “q” satisfy the following conditions:
0≦p≦90; 0≦q≦10; but when q=0, at least one “B” is R⁶}.

In the above formula, R¹ is a sunsubstituted or substituted monovalent hydrocarbon group (except for groups corresponding to R⁶). The unsubstituted monovalent hydrocarbon group is exemplified by methyl, ethyl, propyl, or similar alkyl groups; phenyl, tolyl, xylyl, or similar aryl groups; or aralkyl groups. The substituted monovalent hydrocarbon group is exemplified by 3,3,3-trifluoropropyl, 3,3,4,4,4-pentafluorobutyl, or similar perfluoroalkyl groups; methoxycarbonylpropyl, ethoxycarbonylpropyl, or similar alkoxycarbonylalkyl groups; acetoxypropyl, propionoxypropyl, or similar alkylcarbonyloxyalkyl groups; ethoxyethyl, or similar alkoxyalkyl groups; 3-aminopropyl, 3-(aminoethyl)aminopropyl, or similar aminoalkyl groups; alkylaminocarbonylalkyl, or alkylcarbonylaminoalkyl groups (except for groups corresponding to R⁶). A part of the groups represented by R¹ may be replaced by an alkoxy group or alkoxy groups such as methoxy, ethoxy, or propoxy groups. Among the above, R¹ is preferably the unsubstituted monovalent hydrocarbon group, in particular, the alkyl group, and, more specifically, methyl group.

Among the polyoxyalkylene-modified diorganopolysiloxane represented by average structural formula (3), a polyoxyalkylene-modified diorganopolysiloxane of the following average structural formula (3-1) is preferable:

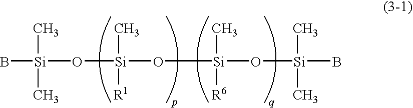

where R¹ is the same as defined above, and R⁶ is a polyoxyalkylene group of the following general formula (4): —R⁷—O—(C₂H₄O)_{d}(C₃H₆O)ₑR⁸ [where R⁷ is an alkylene group with 2 to 8 carbon atoms; R⁸ is a group selected from hydrogen atom, an alkyl group with 1 to 12 carbon atoms, and an organic group of the following formula: —(OC)—R⁹ (where R⁹ is an alkyl group with 1 to 12 carbon atoms)]. R⁸ is preferably hydrogen atom from the viewpoint of surface-active properties. It is also recommended that the aforementioned alkylene group has 2 to 6 carbon atoms, preferably 3 or 4 carbon atoms. It is preferable that the alkyl group represented by R⁹ have 1 to 8 carbon atoms.
“d” and “e” are numbers that satisfy the following conditions: 1≦d≦20; 0≦e≦20; 5≦(d+e)≦40, preferably 8≦(d+e)≦20. “B” designates groups that may be the same or different and are selected from hydroxyl group, R¹ and R⁶; “p” and “q” satisfy the following conditions: 0≦p≦90, preferably 50≦p≦80; 0≦q≦10, preferably 0≦q≦5; when q=0, at least one “B” is R⁶. However, both B can be R⁶, as well.
If the values of “d”, “e”, “p”, and “q” exceed the recommended upper limit, situations where the polyoxyalkylene-modified diorganopolysiloxanes of the average structural formula (3-1) cannot be distinguished from component (a) occur. This leads to decrease in emulsification capability when the inventive composition for the preparation of cosmetics is mixed with water for emulsification, and to decrease in temporal stability of prepared emulsion cosmetics.
There are no special restrictions with regard to the sequence of siloxane units in the aforementioned average structural formulas (3) and (3-1), and the sequence of these units may be random, blocked, or a combination of both, but generally random in copolymers. The same is true for —(C₂H₄O)ₐ(C₃H₆O)_{b}—.

In the polyoxyalkylene-modified diorganopolysiloxanes of average structural formulas (3) and (3-1), the diorganopolysiloxane part has a lower degree of polymerization than the diorganopolysiloxane part of component (a), and the maximal degree of polymerization of the polyoxyalkylene groups thereof is lower than the maximal degree of polymerization of the polyoxyalkylene groups in component (a). It is recommended that the polyoxyalkylene-modified diorganopolysiloxanes of average structural formulas (3) and (3-1) have HLB, which represents hydrophilic and lipophilic balance, in the range of 3 to 10, and preferably 3 to 8. By using the aforementioned polyoxyalkylene-modified diorganopolysiloxane that has a low degree of polymerization and HLB ranging from 3 to 8 in combination with component (a), it becomes possible to improve temporal stability of the oil-in-water emulsion type cosmetics obtained by mixing the inventive composition for the preparation of cosmetics with water.

Component (c), the oil of one or more types, is animal oil, vegetable oil, mineral oil or the like normally used in conjunction with the preparation of cosmetics. If the oil is hydrophobic, then irrespective of its origin, it may be in a solid, semi-solid, or liquid state, as well as be non-volatile, semi-volatile, or volatile. Examples of the oil include hydrocarbon oil, fat, wax, hardened oil, ester oil, higher fatty acid oil, silicone oil, fluorinated oil, or lanolin derivative type oil. The oil is used for imparting lubricity to the skin and the hair, to make the skin soft, and to maintain a moist feeling of the skin.

The aforementioned silicone oil as the oil has hydrophobic properties, and may have a cyclic, linear, or branched molecular structure. Viscosity of this oil at 25° C. may range from 0.65 to 100,000 mm²/s, and preferably is in the range of 0.65 to 10,000 mm²/s. Naturally, this component should not include polyoxyalkylene-modified diorganopolysiloxanes such as components (a) or (b2). Specific examples of the aforementioned silicone oil include octamethylcyclotetrasiloxane, tetramethylcyclopentasiloxane, or a similar cyclic diorganopolysiloxane; hexamethyldisiloxane, octamethyltrisiloxane, decamethyltetrasiloxane, dimethylpolysiloxane capped at both molecular terminals with trimethylsiloxy groups, methylphenylpolysiloxane capped at both molecular terminals with trimethylsiloxy groups, methylalkylpolysiloxane capped at both molecular terminals with trimethylsiloxy groups, or a similar linear-chain diorganopolysiloxane, methyltris(trimethylsiloxy)silane, ethyl tris(trimethylsiloxy)silane, propyltris(trimethylsiloxy)silane, tetrakis[tris(trimethylsiloxy)]silane, phenyltris(methylsiloxy)silane, or a similar branched organopolysiloxane. Of these, preferable are volatile linear dimethylpolysiloxane, branched methylpolysiloxane, and cyclic dimethylpolysiloxane.

Specific examples of cyclic organopolysiloxanes÷include hexamethyl cyclotrisiloxane (D3); octamethyl cyclotetrasiloxane (D4); decamethyl cyclopentasiloxane (D5); dodecamethyl cyclohexasiloxane (D6); 1,1-diethylhexamethyl cyclotetrasiloxane; phenylheptamethyl cyclotetrasiloxane; 1,1-diphenylhexamethyl cyclotetrasiloxane; 1,3,5,7-tetravinyltetramethyl cyclotetrasiloxane; 1,3,5,7-tetramethyl cyclotetrasiloxane; 1,3,5,7-tetracyclohexyltetramethyl cyclotetrasiloxane; tris(3,3,3-trifluoropropyl) trimethyl cyclotrisiloxane; 1,3,5,7-tetra (3-aminopropyl) tetramethyl cyclotetrasiloxane; 1,3,5,7-tetra-(N-(2-aminoethyl) 3-aminopropyl) tetramethyl cyclotetrasiloxane; 1,3,5,7-tetra-(3-mercaptopropyl) tetramethyl cyclotetrasiloxane; 1,3,5,7-tetra-(3-glycidoxypropyl) tetramethyl cyclotetrasiloxane; 1,3,5,7-tetra-(3-methacryloxypropyl) tetramethyl cyclotetrasiloxane; 1,3,5,7-tetra-(3-acryloxypropyl) tetramethyl cyclotetrasiloxane; 1,3,5,7-tetra-(3-carboxypropyl) tetramethyl cyclotetrasiloxane; 1,3,5,7-tetra-(3-vinyloxypropyl) tetramethyl cyclotetrasiloxane; 1,3,5,7-tetra-(p-vinylphenyl) tetramethyl cyclotetrasiloxane; 1,3,5,7-tetra-[3-(p-vinylphenyl) propyl] tetramethyl cyclotetrasiloxane; 1,3,5,7-tetra-[3-(p-isopropenyl benzoylamino) propyl] tetramethyl cyclotetrasiloxane; 1,3,5,7-tetra-(N-methacryloyl-N-methyl-3-aminopropyl) tetramethyl cyclotetrasiloxane; 1,3,5,7-tetra-(N-lauroyl-N-methyl-3-aminopropyl) tetramethyl cyclotetrasiloxane; 1,3,5,7-tetra-(N-acryloyl-N-methyl-3-aminopropyl) tetramethyl cyclotetrasiloxane; 1,3,5,7-tetra-(N,N-bis(methacryloyl)-3-aminopropyl) tetramethyl cyclotetrasiloxane; and 1,3,5,7-tetra-(N,N-bis(lauroyl)-3-aminopropyl) tetramethyl cyclotetrasiloxane. The linear organopolysiloxanes are exemplified by dimethylpolysiloxane having both ends of the molecular chain blocked by trimethylsiloxy groups, methylphenylpolysiloxane having both ends of the molecular chain blocked by trimethylsiloxy groups, copolymers of methylphenylsiloxane and dimethyl siloxane having both ends of the molecular chain blocked by trimethylsiloxy groups, copolymers of methyl (3,3,3-trifluoropropyl)siloxane and dimethyl siloxane having both ends of the molecular chain blocked by trimethylsiloxy groups, αω-dihydroxypolydimethyl siloxane, α,ω-dimethoxypolydimethylsiloxane, tetramethyl-1,3-dihydroxydisiloxane, octamethyl-1,7-dihydroxytetrasiloxane, hexamethyl-1,5-diethoxytrisiloxane, hexamethyldisiloxane, and octamethyltrisiloxane.

Oils other than silicone oils in component (c) are liquid at temperature ranging from 5° C. to 100° C. For instance, hydrocarbon oils are exemplified by ozokerite, squalane, squalene, ceresin, paraffin, paraffin wax, liquid paraffin, pristane, polyisobutylene, polybutene, microcrystalline wax, Vaseline, etc. Animal oils and vegetable oils are exemplified by avocado oil, linseed oil, almond oil, Ericerus pela chabannes wax, perilla oil, olive oil, cacao butter, Kapok tree wax, kaya oil, carnauba wax; cod-liver oil, candelilla wax, beef tallow, hoof oil, cow bone fat, hardened beef tallow, persic oil, spermaceti wax; hardened oils, wheat germ oil, sesame oil, rice germ oil, rice bran oil, sugarcane wax, camellia Kissi seed oil, safflower oil, shear butter, Paulownia oil, cinnamon oil, jojoba wax, shellac wax, turtle oil, soybean oil, tea seed oil, camellia oil, evening primrose oil, corn oil, lard, rape-seed oil, Japanese tung oil, bran wax, germ oil, horse fat, persic oil, palm oil, palm kernel oil, castor oil, hardened castor oil, castor oil fatty acid methyl ester, sunflower oil, grape seed oil, bayberry wax, jojoba oil, macadamia nut oil, yellow beeswax, mink oil, cottonseed oil, cotton wax, Japan wax, sumac kernel oil, montan wax, palm oil, hardened palm oil, cocoglycerides, mutton tallow, peanut oil, lanolin, liquid lanolin, reduced lanolin, lanolin alcohol, hardened lanolin, lanolin acetate, lanolin fatty acid isopropyl ester, lauric acid hexyl ester, POE lanolin alcohol ether, POE lanolin alcohol acetate, lanolin fatty acid polyethylene glycol, POE hydrogenated lanolin alcohol ether, yolk oil, etc.; higher alcohols are exemplified by lauryl alcohol, myristyl alcohol, palmityl alcohol, stearyl alcohol, behenyl alcohol, hexadecyl alcohol, oleyl alcohol, isostearyl alcohol, hexyldodecanol, octyldodecanol, cetostearyl alcohol, 2-decyltetradecynol, cholesterol, phytosterols, POE cholesterol ether, monostearyl glycerin ether (batyl alcohol), monooleyl glyceryl ether (selachyl alcohol), etc.; ester oils are exemplified by ad diisobutyl adipate, 2-hexyldecyl adipate, di-2-heptylundecyl adipate, N-alkylglycol monoisostearate, isocetyl isostearate, trimethylolpropane triisostearate, ethylene glycol di-2-ethylhexanoate, cetyl 2-ethylhexanoate, trimethylolpropane tri-2-ethylhexanoate; pentaerythritol tetra-2-ethylhexanoate, cetyl octanoate; octyl dodecyl gum ester; oleyl oleate; octyl dodecyl oleate; decyl oleate isononyl isononanoate, neopentyl glycol dicaprate, triethyl citrate, 2-ethylhexyl succinate, amyl acetate; ethyl acetate; butyl acetate; isocetyl stearate, butyl stearate, diisopropyl sebacate; 2-ethylhexyl sebacate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid ester, isopropyl myristate, 2-ethylhexyl myristate; octyl dodecyl myristate, 2-hexyldecyl myristate, myristyl myristate, hexyldecyl dimethyloctanoate, ethyl laurate, hexyl laurate, N-lauroyl L-glutamic acid octyl dodecyl ester, diisostearyl malate, etc.; and glyceride oils, such as acetoglyceride, glyceryl triisooctanoate, glyceryl triisostearate, glyceryl triisopalmitate, glyceryl tri(capryl caprate), glyceryl monostearate, glyceryl di-2-heptylundecanoate, glyceryl trimyristate, diglyceryl myristate isostearate, etc.

Component (c) is preferably silicone oils, which are preferably volatile linear-chained dimethylpolysiloxanes, branched methylpolysiloxanes, or cyclic dimethylpolysiloxanes; and more preferably dimethylpolysiloxane capped at both molecular terminals with trimethylsiloxy groups, methylphenylpolysiloxane having both molecular terminals capped with trimethylsiloxy groups, α,ω-dimethoxypolydimethylsiloxane, which have a viscosity at 25° C. in the range of 0.65 to 10,000 mm²/s, or decamethylcyclopentasiloxane (D5). Addition of these silicone oils to the inventive composition for preparation of cosmetics improves temporal stability of oil-in-water emulsion type cosmetics obtained from the composition and imparts a feel of freshness inherent in silicone oils.

Component (c) other than silicone oils is preferably squalane, paraffin or similar hydrocarbon oils, and isononyl isononate, or similar fatty acid ester oils. These oils can be used independently or in combinations with each other, but preferably in combination with the aforementioned silicone oil. The combination of such hydrocarbon oil or fatty acid ester oil with the silicone oil imparts a feel of freshness inherent in silicon oils and moisture retention properties that assists in retention of moisture on the skin and imparts to the skin a feel of smoothness. Another advantage of the above combination is that it does not impair temporal stability of the cosmetics. The cosmetic comprising the composition for the preparation of cosmetics and the aforementioned hydrocarbon oil or fatty acid ester oil and the silicone oil provides conditions for more stable and uniform coating such moisture-retention component onto the skin or hair and improves moisture-holding effect on the skin. Such a cosmetic with combined oil components is superior in its feel of freshness to cosmetics that contain only hydrocarbon oil or only fatty acid ester oil.

Hydrocarbon oils, fats, waxes, hardened oils, ester oils, higher fatty acids, silicone oils, fluorinated oils, and lanolin derivatives as component (c) can be combined with each other in an amount of two or more. The aforementioned specific examples can be combined with each other in an amount of two or more.

Components (a), (b), and (c) that constitute the composition for the preparation of cosmetics of the present invention are mixed preferably in a ratio that facilitates formation of an oil-in-water emulsion and makes it possible to obtain emulsion particles with an average diameter (measured by a laser diffraction/dispersion method for emulsion particles) of less than 10.0 μm (10000 nm). For this purpose, component (a) is preferably present in an amount of 0.85 to 680 parts by mass, and component (b) is preferably present in an amount of 1.4 to 1120 parts by mass per 100 parts by mass of component (c).

If components (a) and (b) are present in an amounts close to the aforementioned lower limit, this will create conditions insufficient for emulsification of component (c) when the composition for the preparation of cosmetics of the present invention is mixed with water. If, on the other hand, components (a) and (b) are present in an amounts close to the aforementioned upper limit, the content of the oil will become too small, and it will be impossible to impart to the obtained cosmetics desired tactile properties. Therefore, the best results can be expected when component (a) is present in an amount of 1.0 to 340 parts by mass, preferably, 1.2 to 68 parts by mass, and component (b) is present in an amount of 1.5 to 560 parts by mass, preferably 1.9 to 112 parts by mass per 100 parts by mass of component (c).

Since the inventive composition for the preparation of cosmetics that comprises a mixture of aforementioned components (a) to (c) is easily emulsified by mixing with water, a stable oil-in-water emulsion with emulsion particles having an average diameter lower than 10 μm can be obtained by mixing the composition with an appropriate amount of water without the use of any high-pressure emulsifier, colloidal mill, colloidal mixer, or similar emulsifiers exerting high shear forces, rather with the use of a simple and well-known mixer or stirrer such as paddle-blade stirrer, propeller stirrer, Henschel mixer, TK Homomixer (the product of Tokushu Kaka Kogyo Co., Ltd.), TK Homodisper (the product of Tokushu Kika Kogyo Co., Ltd.), or similar well known simple mixers.

What is meant here under the term “high-pressure emulsifier” is a high pressure device used for crushing emulsified organopolysiloxane particles in a primary emulsion to create uniform and microscopic emulsified particles therein. In this device, the primary emulsion is subjected to high pressure to become high-speed flows, the high-speed flows are branched, and the branched high-speed flows are collided each other, or the high-speed flows are passed through adjustable microscopic gaps, and are collided to impact rings or rotating bodies, and the organopolysiloxane particles in the primary emulsion are crushed by colliding forces, shear forces, cavitation, turbulence, or ultrasonic waves generated in the aforementioned collisions to become uniform microscopic emulsified particles. Specific examples of such devices include super-high-pressure Gaulin-type homogenizer (the product of Gaulin Co.), high-pressure homogenizer (the product of Izumi Food Machinery Co., Ltd.), high-pressure homogenizer (the product of Rannie Co.), Microfluidizer (the product of Microfluidics Co.), and Nanomizer (the product of Nanomizer Co.).

The inventive composition for the preparation of cosmetics that comprises a mixture of aforementioned components (a), (b), and (c) may further comprise component (d), a biologically allowable hydrophilic medium of one or more types. In addition to aforementioned component (d), the composition can further comprise component (e) water. Dissolving or dispersing components (a) to (c) in component (d), a biologically allowable medium of one or more types, improves degree of transparency of the emulsion, and facilitates the formation of a oil-in-water emulsion having temporal stability and microscopic average particle size, i.e. an average particle size of less than 0.5 μm (as measured by the laser diffraction/dispersion method) when a composition for the preparation of cosmetics comprising components (a), (b), (c) and (d) is mixed with water. It becomes possible to prepare oil-in-water type emulsion cosmetics with higher temporal stability simply.

Component (d), the biologically allowable hydrophilic medium of one or more types, is a liquid that is characterized by excellent compatibility with water at room temperature. Typical biologically allowable hydrophilic medium does not cause irritation or minimal irritation on human skin when incorporated in cosmetics. Specifically it does not impair appearance and/or feel of touch on skin or hair, and does not present a hazard to health even if taken orally in a relatively large quantity. It is preferable that component (d) is a carbinol-containing aliphatic hydrocarbon compound. Specific examples of such component (d) include ethanol, n-propanol, isopropanol, n-butanol, or a similar monovalent lower alcohol; 1,3-butyleneglicol, ethyleneglycol, propyleneglycol, or a similar bivalent alcohol; polyethyleneglycol, dipropyleneglycol, polypropyleneglycol, or a similar polyalkyleneglycol; glycerol, diglycerol, trimethylolpropane, pentaerythritol, sorbitol, or a similar polyalcohol. These media can be used in combination of two or more ones.

When the composition for the preparation of cosmetics of the present invention is for the preparation of oil-in-water emulsion cosmetics and, in particular for the preparation of oil-in-water type emulsion cosmetics that have emulsion particles with an average diameter of less than 0.2 μm (200 nm) (when measured by the laser diffraction/dispersion method), it is preferable that component (d) is ethanol, isopropanol, or 1,3-butyleneglycol. Inclusion of the aforementioned hydrophilic medium makes the liquid mixture more transparent, significantly facilitates dispersion of the composition in water with the use of a conventional stirrer or mixer, and allows to form oil-in-water emulsion type cosmetics with an average particle size of less than 0.2 μm. When the production scale is small, an oil-in-water emulsion type cosmetic with an average particle size of less than 0.2 μm can be prepared, even if the components are mixed manually with the use of a spatula. An advantage of the composition that comprises a mixture of components (a), (b), and (c) is that it possesses self-emulsification properties, and, therefore, preparation of a stable oil-in-water type emulsion cosmetic from the aforementioned composition practically does not require any preliminary investigation of the emulsification conditions.

The most desirable component (d) is ethanol. From the viewpoint of the self-emulsification properties of the composition for the preparation of cosmetics of the present invention, it is recommended to mix ethanol with the aforementioned biologically allowable hydrophilic medium other than ethanol in a mass ratio of 5/5 to 9.9/0.1. It is most preferable from the viewpoint of self-emulsification properties that the mass ratio of ethanol to the biologically allowable hydrophilic medium other than ethanol is 6/4 or more.

Preferable proportion of the components (a), (b), (c), and (d) in the mixture that forms the composition for the preparation of cosmetics of the present invention is such that mixture thereof becomes transparent liquid and has excellent emulsification properties, namely, forms an oil-in-water type emulsion with microscopic emulsion particles when the mixture is mixed with water, where the aforementioned particles have an average diameter (measured by the laser diffraction/dispersion method) less than 5 μm (500 nm), preferably less than 0.2 μm (200 nm).

From the above viewpoint, it is recommended to contain component (d) in an amount of 20.0 to 98.4 mass %, and preferably, 40.0 to 98.4 mass % of the total mass of the composition for the preparation of cosmetics. For better temporal stability of the prepared oil-in-water type emulsion, it is recommended to contain component (d) in the range of 55.0 to 98.4 mass %.
Even if component (d) is contained in an amount of less than the recommended lower limit, it is possible to prepare oil-in-water emulsion type cosmetics easily as mentioned before In that case, however, preparing oil-in-water emulsion type cosmetics with small particle sizes such as an average diameter of less than 0.5 μm may require the use of a high-pressure emulsifier, colloidal mill or mixer of the type that develop high shear forces, and preliminary investigations may be required for finding the most optimal emulsification conditions.

When component (d) is contained in the above range, for preparing oil-in-water type emulsion cosmetic with emulsion particles having an average diameter of less than 0.2 μm (200 nm) when measured by the laser diffraction/dispersion method, and with a property of excellent temporal stability, it is recommended that component (a) is contained in an amount of 0.9 to 90 pats by mass and preferably 0.9 to 45 parts by mass; component (b) is contained in an amount of 0.6y to 120 parts by mass and preferably 0.6 to 60 parts by mass; and component (c) is contained in an amount of 1.1 to 150 pats by mass, and preferably 1.1 to 75 parts by mass per 100 parts by mass of component (d).

While maintaining the above ratios of components (a), (b), and (c) with respect to 100 parts by mass of component (d), it is also recommended that component (a) be contained in an amount of 4 to 300 parts by mass, preferably 4 to 200 parts by mass, and component (b) be contained in an amount of 5 to 480 parts by mass, preferably 5 to 320 parts by mass, per 100 pats by mass of component (c).

If components (a) and (b) are contained in amounts less than the aforementioned lower limit, the oil-in-water type emulsion cosmetics prepared by mixing the composition for the preparation of cosmetics with water may not possess sufficient temporal stability, and if, on the other hand, the aforementioned components are contained in amounts exceeding the aforementioned upper limit, the prepared oil-in-water type emulsion cosmetics may not acquire desired feel to touch.

With regard to the content of each component in the inventive composition for the preparation of cosmetics comprising a mixture of components (a) to (d), it is recommended that component (a) of Claim 1 is contained in an amount of 1 to 25 mass %, component (b) of Claim 1 is contained in an amount of 0.5 to 25 mass %, component (c) of Claim 1 is contained in an amount of 0.1 to 40.0 mass %, component (d) of Claim 2 is contained in an amount of 20.0 to 98.4 mass %, and component (c) is contained in an amount of not exceeding component (d).

In order to obtain emulsion particles with an average diameter measured by a laser diffraction/dispersion method less than 1.0 μm (100 nm), it is preferable that the content of component (d) is 55.0 to 98.4 mass % of the total mass of the composition for the preparation of cosmetics, component (a) is contained in an amount of 0.9 to 30 pats by mass, component (b) is contained in an amount of 0.6 to 40 parts by mass, and component (c) is contained in an amount of 1.1 to 50 parts by mass per 100 parts by mass of component (d), and component (d) is ethanol alone or a mixture of ethanol and the aforementioned biologically allowable hydrophilic medium other than ethanol (the mass ratio of the ethanol to the aforementioned medium should be not less than 6/4), component (b) is composed of (b1) an ionic surfactant of one or more types and (b2) a nonionic surfactant of one or more types, and component (c) is a hydrophobic silicone oil having a viscosity of 0.65 mPa·s to 100,000 mPa·s at 25° C.

Furthermore, the inventive composition for the preparation of cosmetics that comprises a mixture of components (a), (b), (c), and (d) may further comprise (e) water. Component (e), the water, should not contain components harmful to humans and should be clean. Water may be exemplified by tap water, purified water, and mineral water. The inventive composition for the preparation of cosmetics that contains a biologically allowable hydrophilic medium of one or more types that is component (d), in particular, ethanol or a similar lower alcohol, is highly flammable. However, compounding with water reduces flammability and provides higher safety during manufacturing, storage, and transportation. Furthermore, water-soluble components, such as water-soluble ionic surfactants, can be preliminarily dispersed in water and then compounded with the composition for the preparation of cosmetics of the present invention.

In case the cosmetic of the present invention is a cosmetic, particularly an oil-in-water type emulsion cosmetic which is prepared by mixing the inventive composition for the preparation of cosmetics containing (e) water with additional water, water preliminarily contained in the composition for the preparation of cosmetics and water added during the manufacturing process can not be distinguished in the cosmetic.

However, water (e) contained in the composition decreases self-emulsification properties of the composition for the preparation of cosmetics of the present invention, increases average diameter of the emulsion particles, and reduces temporal stability of the oil-in-water type emulsion cosmetic. Therefore, component (e) should be contained in an amount not exceeding the content of component (d). In general, the content of component (e) is preferably less than 40.0 mass %, more preferably less than 30 mass %, of the entire composition for the preparation of cosmetics. If the content of component (e) exceeds the aforementioned upper limit, the composition for the preparation of cosmetics itself contains much water, and this will create mutual compatibilization of the components in the mixture and conditions of incomplete emulsification, and may impair self-emulsification properties which is one of advantageous effects of the present invention.

Preferable contents of each component in the composition for the preparation of cosmetics, which comprise a mixture of components (a), (b), (c), and (d), are contents such that the mixture becomes a transparent liquid, the composition possesses self-emulsification properties and, when mixed with water, can be easily turned into an oil-in-water type emulsion having microscopic emulsion particles with an average diameter less than 0.5 μm (500 nm), and preferably less than 0.2 μm (200 nm), when measured by the laser diffraction/dispersion method.

Specifically, it is preferable that component (a) is contained in an amount of 0.9 to 90 parts by mass, component (b) is contained in an amount of 0.6 to 120 parts by mass, and component (c) is contained in an amount of 1.1 to 150 parts by mass per 100 parts by mass of component (d). On the other hand, component (d) is contained in an amount of 20.0 to 98.4 mass % of the entire composition for the preparation of cosmetics; component (e) is contained in an amount less than the content of component (d) and not more than 40.0 mass % of the entire composition for the preparation of cosmetics. The preferable contents of components (a) and (b) per 100 parts by mass of component (c), and the preferable content of component (d) per sum of components (a), (b), (c), and (d) should be the same as defined earlier.

Thus, the individual components are contained preferably in the following amounts per total weight of the composition for the preparation of cosmetics that comprises a mixture of components (a) to (e): component (a) is contained in an amount of 1 to 25 mass %; component (b) is contained in an amount of 0.5 to 25 mass %; component (c) is contained in an amount of 0.1 to 40 mass %; component (d) is contained in an amount of 20.0 to 98.4 mass %; component (e) is contained in an amount less than component (d) and not more than 40 mass % of the entire composition for the preparation of cosmetics; and component (c) is contained in an amount not more than the content of component (d).

The inventive composition for the preparation of cosmetics that comprises a mixture of aforementioned components (a) to (c), and the composition for the preparation of cosmetics that comprises a mixture of components (a) to (d) can be prepared by loading the aforementioned components into a container and stirring them with the use of a stirrer. If necessary, a specific stirring device or mixing device can be used for this purpose.

The composition for the preparation of cosmetics that comprises a mixture of components (a) to (d) can be easily prepared by first mixing components (a) to (c) and then adding component (d) and mixing it with the premixed components.
The composition for the preparation of cosmetics that comprises a mixture of components (a) to (e) can be easily prepared by first mixing components (a) to (d) and then adding component (e) and mixing it with the premixed components, or by first mixing components (a) to (c) and then adding to the first mixture a premixed components (d) and (e). The sequence of addition of various components and types of stirring and mixing devices do not exert a significant influence on the temporal stability and diameter of particles of the oil-in-water type emulsion cosmetics obtained by mixing the inventive for the preparation of composition with water.

The composition for the preparation of cosmetics that comprises a mixture of components (a) to (c), the composition for the preparation of cosmetics that comprises a mixture of components (a) to (d), and the composition for the preparation of cosmetics that comprises a mixture of components (a) to (e), may further contain (i) a water-soluble polymer, (j) an antiseptic agent, (k) an antibacterial agent, or (n) an antioxidant, which are described below, or plural these agents within the limits that are not harmful to the properties thereof. (i) a water-soluble polymer is added to increase viscosity or to improve stability of the compositions; (j) an antiseptic agents and (k) an antibacterial agent are added to prevent bacterial spoilage; and (n) an antioxidant is added to prevent oxidation of the non-silicone-type oils.

The cosmetic of the present invention is characterized by containing the composition for the preparation of cosmetics comprising a mixture of components (a) to (c), the composition for the preparation of cosmetics comprising a mixture of components (a) to (d), or the composition for the preparation of) cosmetics comprising a mixture of components (a) to (e). In particular, this cosmetic is characterized by being the oil-in-water type emulsion cosmetic and a skin cosmetic.

The cosmetic of the present invention comprises the inventive composition for the preparation of cosmetics and other component normally added to conventional cosmetics. Components normally added to conventional cosmetics are exemplified by (f) a moisture-retaining agent, one to three types of (g) silicones selected from gum-type silicone, silicone resin, or a silicone elastomer powder, (h) an ultraviolet-ray blocker, (i) a water-swellable mineral clay or water-soluble polymer, (j) an antiseptic, (k) an antibacterial agent, (l) a biologically active component, (m) a pH adjuster, (n) an antioxidant (anti-oxidation agent), (o) water, (p) a solvent, (q) a chelating agent, (r) a fragrance, or (s) a coloring agent. One or several types of such other components are selected with reference to the type, use or application, properties, and form of the cosmetic, and to mix with the composition for the preparation of cosmetics of the present invention.

There are no special restrictions with regard to amounts of the composition for the preparation of cosmetics of the present invention and the components that are normally added to cosmetics in the cosmetic of the present invention, and various amounts are selected with reference to types, properties, use or application, and forms of the cosmetic. For example, in a cosmetic consisting of the composition for the preparation of cosmetics of the present invention, a coloring agent and fragrance, the composition for the preparation of cosmetics of the present invention can constitute more than 99 mass %. Since the aforementioned composition for the preparation of cosmetics of the present invention can be easily emulsified and turned into an oil-in-water type emulsion having microscopic emulsion particles without using any specific emulsifier, and the resulting oil-in-water type emulsion cosmetic has superior temporal stability, it is most suitable for manufacturing oil-in-water type emulsion cosmetics. In this case, it is recommended to contain the composition for the preparation of cosmetics of the present invention in an amount of 5 to 50 mass % of the inventive cosmetic.

Desirable amounts of components (a), (b), and (c), as well as of components (a), (b), (c), and (d), or components (a), (b), (c), (d), and (e) in the cosmetic of the present invention, and, in particular, in the oil-in-water type emulsion cosmetic, are amounts obtained by multiplying the desirable amounts of respective components in the composition for the preparation of cosmetics by the concentration of the composition for the preparation of cosmetics of the present invention in the cosmetic, in particular, oil-in-water type emulsion cosmetic.

Component (f), the moisture-retaining agent, that is normally added to cosmetics, and is added to the composition for the preparation of cosmetics of the present invention is preferably a compound which is normally added to cosmetics and pharmaceutical products, such as glycol, xylitol, maltitol, hyaluronic acid, chondroitin sulfate, carboxylate acid salts of pyrrolidone, polyoxyethylene methylglucoside, and polyoxypropylene methylglucoside, etc. Such moisture-retaining agent, which is used for improving a feel of touch of cosmetics, is added in an amount not detrimental to the effects of the present invention. Component (d) such as 1,3-butylene glycol, ethylene glycol, polyethylene glycol, propylene glycol, dipropylene glycol, polypropylene glycol, glycerin, trimethylpropane, pentaerythritol, sorbitol, or the like contributes to enhancing moisture-retaining properties in some cases.

Component (g), the silicones, that are one of components normally added to cosmetics and are added to the composition for the preparation of cosmetics of the present invention is one to three selected from the group consisting of a gum-like silicone, a silicone resin, or a silicone elastomer powder.

The gum-like silicone is a linear-chain diorganopolysiloxane of ultra-high degree of polymerization, which is called also a silicone gum or organopolysiloxane gum. A typical example of the above gum-like silicone is represented by the following formula:

(CH₃)₃SiO{(CH₃)₂SiO}ₐ{(CH₃)R³SiO}_{b}Si(CH₃)₃ (where R³ is preferably selected from a vinyl group, phenyl group, an alkyl group with 6 to 20 carbon atoms, an aminoalkyl group with 3 to 15 carbon atoms, a perfluoroalkyl group with 3 to 15 carbon atoms, and an alkyl group containing quaternary ammonium salt with 3 to 15 carbon atoms; the subscript “a” preferably being in the range of from 3 to 6000, the subscript “b” being in the range of 0 to 1,000, and (a+b) being in the range of 3000 to 6000).

The silicone resin is an organopolysiloxane with highly branched molecular structure, a net-like molecular structure, or a cage-like molecular structure. It may be liquid or solid at room temperature, and within the limits not contradictory to the objects of the present invention, it may be any silicone resin normally added to conventional cosmetics.

The solid silicone resin is exemplified by MQ resins, MDQ resins, MTQ resins, MDTQ resins, TD resins, TQ resins, or TDQ resins comprising arbitrary combinations of triorganosiloxy unit (M unit) (where the organic group is methyl group alone, or methyl group with vinyl group or with phenyl group), diorganosiloxy unit (D unit) (where organo group is methyl group alone, or methyl group in combination with vinyl group or phenyl group), monoorganosiloxy unit (T unit) (where the organic group is methyl group, vinyl group, or phenyl group). Other examples are trimethyl siloxysilicic acid, polyalkyl siloxysilicic acid, trimethyl siloxysilicic acid that contains dimethylsiloxy units, or alkyl (perfluoroalkyl) siloxysilicic acid. The aforementioned silicone resins are preferably oil soluble, and most preferably, are soluble in octamethyl tetrasiloxane (D4) or tetramethyl cyclopentasiloxane (D5).

The aforementioned gum-like silicone or silicone resin can be added to the composition for the preparation of cosmetics as it is, or as a solution formed by dissolving in a volatile silicone oil, non-volatile silicone oil, volatile hydrocarbon oil, or non-volatile hydrocarbon oil. The amount of such gum-like silicone or silicone resin is preferably in the range of from 0.1 to 20 mass %, and even more preferably, in the range of from 1 to 10 wt % based on the total amount of the cosmetic. In addition, to obtain cosmetics having high adhesive properties to the skin an amount of the gum-like silicone or silicone resin is preferably in the range of from 50 to 500 parts by weight per 100 parts by weight of the composition for the preparation of cosmetics of the present invention.

The silicone elastomer powder comprises a cross-linked product of a linear-chain diorganopolysiloxane that may have various shapes such as spherical, oblate granular, or irregular shape, or may comprise shapeless oil dispersions. In the present invention, it is preferable to use a silicone elastomer powder having a primary spherical shape of particles, wherein the mean primary particle size is in the range of from 0.1 to 50 μm, measured by laser diffraction/scattering methods and/or obtained by observation under an electron microscope. In addition, the silicone elastomer constituting the silicone elastomer powder has a Type A durometer hardness of preferably not exceeding 80, and, more preferably not exceeding 65 according to JIS K 6253 “Method for determining hardness of vulcanized rubber or thermoplastic rubber”.

Such silicone elastomer powder can be prepared by curing a composition that comprises a diorganopolysiloxane with two or more alkenyl groups, an alkylhydrogen polysiloxane that contains two or more silicon-bonded hydrogen atoms, and a hydrochloric acid in an emulsified state or sprayed state. Typical example of the alkenyl-containing diorganopolysiloxane is a dimethylpolysiloxane having both molecular terminals capped with dimethylvinylsiloxy groups. The above diorganopolysiloxane may be replaced by an organic compound having in one molecule two or more vinyl or allyl groups, such as α,ω-alkenyl diene, glycerin triallyl ether, polyoxy alkenylated glycerin triallyl ether, trimethylolpropane triallyl ether, polyoxy alkynylated trimethylolpropane triallyl ethers, etc.

Such silicone elastomer powders are described, e.g., in Kokai Nos. H02-243612, H08-12545, H08-12546, H08-12524, H09-241511, H10-36219, H11-193331, 2000-281523 etc. There are commercial products of Dow Corning Toray Co., Ltd. such as Trefil E-505, 506, 507, 508 etc. belonging to Trefil E-series. These silicone elastomer powders correspond to cross-linked silicone powders listed in the “Standards for Various Types of Cosmetic Ingredients”.

In addition, the surface of these silicone elastomer powders may be treated with a treating agent. Examples of the treating agent include methylhydrogenpolysiloxane, silicone resins, metal soaps, silane coupling agents, silica, titanium oxide and other inorganic oxides, perfluoroalkylsilanes, perfluoroalkylphosphoric acid ester salts and other fluorine compounds.

The silicone elastomer powder may be added to the cosmetic as a paste-like substance obtained by mixing with an oil, or in the form of an aqueous dispersion. More specifically, the paste-like substance can be prepared by mixing the silicone elastomer powder with a liquid component at room temperature such as oil selected from ester oils, hydrocarbon oils, higher alcohols, vegetable oils, or animal fats, and the dispersion can be prepared by dispersing the silicone elastomer powder into water that contains an emulsification agent by applying a mechanical shear force.

While the spherical silicone elastomer powder often has particle sizes exceeding 10 μm and have been difficult to incorporate into aqueous composition stably, but it is possible to form a stable emulsion by incorporating the spherical silicone elastomer powder into the composition for the preparation of cosmetics of the present invention.

The amount of the spherical organopolysiloxane elastomer powder is preferably 0.1 to 30 mass % relative to the mass of the skin cosmetic of the present invention. When it is less than the above-mentioned lower limit, decreased water slippage becomes a problem, and when it exceeds the above-mentioned upper limit, the skin cosmetic becomes too sticky.

Although silicone-modified organic polymers are not conventional components for cosmetics, they also can be added to the composition for the preparation of cosmetics of the present invention. Examples include a polydimethylsiloxane-graft type acrylic copolymer, and, in particular, a carboxysiloxane dendrimer graft type acrylic copolymer disclosed in Kokai 2000-0632245 and an acrylic copolymer having a carboxysiloxane dendrimer structure and containing fluorinated organic groups disclosed in Kokai 2003-226611.

Component (h), the UV-ray protective component, that normally added to cosmetics and is added to the composition for the preparation of cosmetics may be of an organic type or inorganic type.

The inorganic UV-ray protective component is exemplified by inorganic powder pigments, metal powder pigments, etc. such as UV light dispersers including titania, zinc oxide, cerium oxide, titanium suboxide, iron-doped titanium oxides and other metal oxides; iron hydroxides and other metal hydroxides, platy iron oxide, aluminum flake, and other metal flakes; silicon carbide and other ceramics. Among them, at least one type of material selected from metal oxide particulates or metal hydroxide particulates with a mean particle size in the range of from 1 to 100 nm is particularly preferable. These powders are preferably surface-treated using conventional surface-treatment techniques including, e.g., fluorine compound treatment (perfluoroalkyl phosphate treatment, perfluoroalkylsilane treatment, perfluoropolyether treatment, fluorosilicone treatment, and fluorinated silicone resin treatment are preferable), silicone treatment (methylhydrogenpolysiloxane treatment, dimethylpolysiloxane treatment, and vapor-phase tetramethyltetrahydrogencyclotetrasiloxane treatment are preferable), silicone resin treatment (trimethylsiloxysilicic acid treatment is preferable), pendant treatment (the method of adding alkylchains etc., after vapor-phase silicone treatment), silane coupling agent treatment, titanate coupling agent treatment, silane treatment (alkylsilane treatment and alkylsilazane treatment are preferable), oil solution treatment, N-acylated lysine treatment, polyacrylic acid treatment, metal soap treatment (stearic acid salts treatment and myristic acid salts treatment are preferable), acrylic resin treatment, metal oxide treatment, etc., and, treatment using a combination of such treatments. For instance, the surface of the titanium oxide particulate is coated with silicon oxide, alumina or other metal oxides, the treated powder surface is treated with an alkylsilane. The total amount of material used for surface treatment is preferably in the range of from 0.1 to 50 mass % based on the mass of the powder.

The organic UV-ray protective component is exemplified by homomethyl salicylate, octyl salicylate, triethanolamine salicylate, and other salicylic acid type compounds; para-aminobenzoic acid, ethyldihydroxypropyl para-aminobenzoate, glyceryl para-aminobenzoate, octyl dimethyl para-aminobenzoate, amyl para-dimethylaminobenzoate, 2-ethylhexyl para-dimethylaminobenzoate, and other PABA-type compounds; 4-(2-β-glucopyranosyloxy) propoxy-2-hydroxybenzophenone, dihydroxy dimethoxy benzophenone, sodium dihydroxydimethoxybenzophenone disulphonate, 2-hydroxy-4-methoxybenzophenone, hydroxymethoxybenzophenone sulfonic acid and its trihydrates, sodium hydroxymethoxybenzophenone sulfonate, 2-hydroxy-4-methoxybenzophenone-5-sulfuric acid, 2,2′-dihydroxy-4-methoxy benzophenone, 2,4-dihydroxybenzophenone, 2,2′,4,4′-tetrahydroxy benzophenone, 2,2′-dihydroxy-4,4′-dimethoxy benzophenone, 2-hydroxy-4-N-octoxy benzophenone, and other benzophenone-type compounds; 2-ethylhexyl para-methoxycinnamate (also called octyl para-methoxycinnamate), glyceryl di-para methoxycinnamate, mono-2-ethylhexanoatemethyl 2,5-diisopropylcinnamate, 2,4,6-tris[4-(2-ethylhexyloxycarbonyl) anilino]-1,3,5-triazine, methyl bis(trimethylsiloxy) silylisopentyl trimethoxycinnamate isoproyl para-methoxycinnamate/diisopropylcinnamic acid ester mixture, p-methoxyhydrocinnamic acid diethanolamine salt, and other cinnamic acid type compounds; 2-phenyl-benzimidazole-5-sulfuric acid, 4-isopropyldibenzoylmethane-4-tert-butyl-4′-methoxydibenzoylmethane, and other benzoyl methane type compounds; 2-cyano-3,3-diphenylpropane-2-enoic acid 2-ethylhexyl ester (also called octocrylene), 2-ethylhexyl dimethoxybenzylidene oxoimidazolidinepropionate, 1-(3,4-dimethoxyphenyl)-4,4-dimethyl-1,3-pentanedione, cinoxate, methyl-o-aminobenzoate, 2-ethylhexyl-2-cyano-3,3-diphenyl acrylate, 3-(4-methylbenzylidene)camphor, octyltriazone, 2-ethylhexyl 4-(3,4-dimethoxyphenylmethylene)-2,5-dioxo-1-imidazolidine propionate, as well as polymeric derivatives thereof and silane derivatives thereof, etc.

Furthermore, a polymer powder containing the above-mentioned organic UV-ray protective component inside thereof can be used. The polymer powder may be hollow or not, with its mean primary particle size being in the range of from 0.1 to 50 and its particle size distribution being either broad or sharp. Suggested polymer types include acrylic resins, methacrylic resins, styrene resins, polyurethane resins, polyethylene, polypropylene, polyethylene terephthalate, silicone resins, nylons, acrylamide resins, etc. Polymer powders containing the organic UV-ray protective component in the range of from 0.1 to 30 mass % relative to the mass of the powder are preferable, and a polymer powder containing 4-tert-butyl-4′-methoxydibenzoyl methane, which is a UV-A absorber, is particularly preferable.

At least one UV-ray protective components selected from the group consisting of titanium oxide particulate, zinc oxide particulate, 2-ethylhexyl para-methoxycinnamate, 4-tert-butyl-4′-methoxydibenzoyl methane, and benzophenone-type UV absorbers are preferably used since they are commonly used, easy to obtain, and have excellent UV light-protective effects. Using inorganic and organic UV-ray protective components together is especially preferable, and using a UV-ray protective component designed for UV-A and a UV-ray protective component designed for UV-B in combination is even more preferable.

The amount of the inorganic and/or organic UV-ray protective component in the cosmetic of the present invention is preferably in the range of from 0.1 to 60 mass %, and particularly preferably, in the range of from 3 to 40 mass %, based on the total mass of the cosmetic. Furthermore, the amount of the inorganic UV-ray protective component is preferably in the range of from 0.1 to 30 mass % based on the mass of the cosmetic, and the amount of the organic UV-ray protective component is preferably in the range of from 0.1 to 20 mass % based on the mass of the cosmetic.

Component (i), the water-soluble high polymer and water-swellable mineral clay, that are normally added to cosmetics and are added to the composition for the preparation of cosmetics of the present invention are used for increasing viscosity, improving temporal stability, and enhancing feel of use of the water-containing cosmetic or oil-in-water type emulsion cosmetic.

It is preferable that the water-soluble polymer and water-swellable mineral clay are dissolved or dispersed in water to prepare a uniform aqueous solution or dispersion, and then the prepared solution or dispersion is mixed with cosmetic raw material compositions or with other cosmetic components.
The water-soluble polymer may be of an amphoteric, cationic, anionic, or a nonionic type. The water-soluble polymers and water-swellable mineral clay can be used together, or two or more water-soluble polymers can be used in combination.

The amphoteric water-soluble polymer is exemplified by amphoteric starch, dimethyl diallyl ammonium chloride derivatives (e.g., copolymers of acrylamide, acrylic acid, and dimethyl diallyl ammonium chloride, copolymers of acrylic acid and dimethyl diallyl ammonium chloride), methacrylic acid derivatives (e.g., copolymers of alkyl methacrylate and N-methacryloyloxyethyl-N,N-dimethylammonium-α-methylcarboxy betaine, and polymethacryloylethyl dimethylbetaine).

The cationic water-soluble polymer is exemplified by quaternary nitrogen-modified polysaccharides (e.g., cation-modified cellulose, cation-modified hydroxyethyl cellulose, cation-modified guar gum, cation-modified locust bean gum, cation-modified starch, etc.), dimethyl diallyl ammonium chloride derivative (e.g., a copolymer of dimethyl diallyl ammonium chloride and acrylamide polydimethylmethylene piperidinium chloride, etc.), vinylpyrrolidone derivatives (e.g., a salt of copolymer of vinylpyrrolidone and dimethylaminoethyl methacrylic acid copolymer, a copolymer of vinylpyrrolidone and methacrylamidopropyl trimethylammonium chloride, a copolymer of vinylpyrrolidone and methylvinylimidazolium chloride, etc.), and methacrylic acid derivatives (e.g., a copolymer of methacryloyl ethyldimethyl betaine, methacryloylethyltrimethyl ammonium chloride, and 2-hydroxyethyl methacrylate, and a copolymer of methacryloyl ethyldimethyl betaine, methacryloyl ethyl trimethylammonium chloride, and methoxypolyethylene glycol methacrylate copolymer, etc.).

The anionic water-soluble polymer is exemplified by polyacrylic acid or its alkali metal salts, polymethacrylic acid or its alkali metal salts, hyaluronic acid or its alkali metal salts, acetylated hyaluronic acid or its alkali metal salts, water-soluble polymers of aliphatic carboxylic acids such as hydrolyzate of methylvinyl ether-maleic anhydride copolymer or their metal salts, carboxymethylcellulose or its alkali metal salts, a copolymer of methylvinyl ether and maleic acid half ester, acrylic resin alkanolamine solutions, or carboxyvinyl polymers.

The nonionic water-soluble polymer is exemplified by polyvinyl pyrrolidone, highly polymerized polyethylene glycol, a copolymer of vinylpyrrolidone and vinyl acetate copolymer, a copolymer of vinylpyrrolidone and dimethylaminoethyl methacrylate, a copolymer of vinyl caprolactam, vinylpyrrolidone, and dimethylaminoethyl methacrylate, cellulose or its derivatives (e.g., methyl cellulose, ethyl cellulose, hydroxyethyl cellulose, hydroxypropyl cellulose, and carboxymethyl cellulose), keratin, collagen or their derivatives, calcium alginate, pullulan, agar-agar, gelatin, tamarind seed polysaccharides, xanthan gum, carrageenan, high-methoxyl pectin, low-methoxyl pectin, guar gum, pectin, gum arabic, crystalline cellulose, arabinogalactan, gum Karaya, tragacanth gum, alginic acid, albumin, casein, curdlan, gellan gum, dextran, quince seed gum, tragranth gum, chitin-chitosan derivatives, starches (rice, corn, potato, and wheat, etc.), and other natural polymer compounds.

The water-swellable clay mineral a type of colloid-containing aluminum silicate with a three-layer structure, exemplified by the following formula:

(X,Y)₂₋₃(Si,Al)₁₀(OH)₂Z_{1/3}·nH₂O

(where X is Al, Fe (III), Mn (III), or Cr (III); Y is Mg, Fe (I), Ni, Zn, or Li; and Z is K, Na, or Ca).
Such water-swellable clay mineral is specifically exemplified by bentonite, montmorillonite, pyderite, nontronite, saponite, hectorite, and magnesium aluminum silicate, which may be either natural or synthetic clay minerals.

The amount of such component (i) is preferably 0.001 to 5.0 mass %, and more preferably, 0.01 to 3 mass % of the cosmetic of the present invention. When the amount is below 0.001 mass %, the above-mentioned lower limit, the thickening effect will be insufficient, and if the amount exceeds 5 mass %, the cosmetic will become too viscous and will have an impaired feel of use.

Component (j), the antiseptic agent, that is normally added to conventional cosmetics and that can be added to the cosmetic of the present invention is exemplified by paraoxybenzoic acid alkyl ester, benzoic acid, sodium benzoate, sorbic acid, potassium sorbate, phenoxyethanol, etc.

In addition, component (k), the antibacterial agent, that is normally added to conventional cosmetics and that can be added to the cosmetic of the present invention are exemplified by benzoic acid, salicylic acid, carbolic acid, sorbic acid, paraoxybenzoic acid alkyl esters, parachlormethacresol, hexachlorophene, benzalkonium chloride, chlorhexidine chloride, trichlorocarbanilide, trichlosan, light-sensitive ingredients, phenoxyethanol, methylisothiazolinone, etc. These compounds should be used in amounts sufficient to prevent decay of the cosmetic.

Component (l), the biologically active component, that is normally added to conventional cosmetics and that can be added to the cosmetic of the present invention is a substance that impart a certain physiological activity to the skin when applied to the skin. Such component is exemplified by antiinflammatory agents, age inhibitors, pore-tightening agents, hair-growing agents, moisture-retaining agents, blood-circulation-accelerating agents, desiccants, algefacient agents, calorifacient agents, vitamins, amino-acids, wound healing-accelerating agents, irritation reducers, analgesics, cell activators, enzymatic ingredients, etc. Among them, ingredients such as natural vegetable extracts, seaweed extracts, and herbal medicines are particularly preferable. In the present invention, it is preferable to add one, two, or more types of these physiologically active ingredients.

Examples of these biologically active component ingredients are as follow: Angelica keiskei extract, avocado extract, Hydrangea serrata extract, Althea extract, Arnica extract, aloe extract, apricot extract, apricot kernel extract, Gingko biloba extract, Fennel fruit extract, Turmeric root extract, Oolong tea extract, Rosa multiflora extract, Echinacea angustifolia leaf extract, Scutellaria baicalensis root Extract, Phellodendron amurense extract, Coptis rhizome extract, Hordeum vulgare seed extract, Hypericum perforatum extract, Lamium album extract, Nasturtium officinale extract, orange extract, dried sea water solution, seaweed extract, hydrolyzed elastin, hydrolyzed wheat fines, hydrolyzed silk, Chamomile extract, carrot extract, Artemisia capillaris flower extract, Licorice extract, Karkade extract, Pyracantha fortuneana extract, kiwi extract, Cinchona extract, cucumber extract, guanosine, Gardenia florida extact, Sasa veitchii extract, Sophora angustifolia extract, walnut extract, grapefruit extract, Clematis vitalba leaf extract, chlorella extract, Morus alba root extract, Gentiana lutea extract, black tea extract, yeast extract, burdock extract, fermented rice bran extract, rice germ oil, Comfrey extract, collagen, Vaccinum vitis idaea extract, Asiasarum root extract, Bupleurum falcatum extract, umbilical extract, Salvia extract, Soapwort extract, Sasa bamboo grass extract, Crataegus cuneata fruit extract, Zanthoxylum piperitum extract, Shiitake extract, Rehmannia root extract, Lithospermum erythrorhizone root extract, Perilla ocymoides extract, Tilia cordata flower extract, Spiraea ulmaria extract, Paeonia albiflora extract, Acorns calamus root extract, Betula alba extract, Equisetum arvense extract, Hedera helix extract, Crataegus oxyacantha extract, Sambucus nigra extract, Achillea millefolium extract, Mentha piperita leaf extract, Sage extract, Malva sylvestris extract, Cnidium officinale root extract, Swertia japonica extract, Soybean extract, Zizyphus jujuba fruit extract, thyme extract, tea extract, Eugenia caryophyllus flower extract, Imperata cylindrica extract, Citrus unshiu Marc extract, Angelica root extract, Calendula officinalis extract, Prunus persica stone extract, Citrus aurantium peel extract, Houttuynia cordata extract, tomato extract, natto extract, carrot extract, garlic extract, Rosa canina fruit extract, Hibiscus extract, Ophiopogon extract, Nelumbo nucifera extract, parsley extract, honey, Witch hazel extract, Parietaria officinalis extract, Isodon trichocarpus extract, bisabolol, Eriobotrya japonica extract, Coltsfoot flower extract, Petasites japonicus extract, Poria cocos extract, Butcher's broom extract, grape extract, propolis, Luffa cylindrica fruit extract, Safflower flower extract, peppermint extract, Tillia miquellana extract, Paeonia suffruticosa root extract, hops extract, Pinus sylvestris cone extract, horse chestnut extract, Japanese skunk-cabbage extract, Sapindus mukurossi peel extract, Melissa extract, peach extract, Centaurea cyanus flower extract, Eucalyptus extract, Saxifraga sarementosa extract, Citrus junos extract, Coix seed extract, Artemisia princeps extract, lavender extract, apple extract, lettuce extract, lemon extract, Astragalus sinicus extract, rose extract, rosemary extract, Roman chamomile extract, and royal jelly extract.

In addition, examples of the biologically active component are as follow: deoxyribonucleic acid, mucopolysaccharides, sodium hyaluronate, sodium chondroitin sulfate, collagen, elastin, chitin, chitosan, hydrolyzed eggshell membrane and other biopolymers etc.; glycine, valine, leucine, isoleucine, serine, threonine, phenylalanine, arginine, lysine, aspartic acid, glutamate, cystine, cysteine, methionine, tryptophan and other amino acids; estradiol, ethenyl estradiol and other hormones; phingolipids, ceramides, cholesterol, cholesterol derivatives, phospholipids and other oily ingredients; ε-aminocaproic acid, glycyrrhizinic acid, lysozyme chloride, guaiazulene, hydrocortisone, allantoin, tranexamic acid, azulene and other anti-inflammatory agents; vitamins A, B2, B6, C, D, and E, calcium pantothenate, biotin, nicotinic-acid amide, vitamin C ester, and other vitamins; allantoin, diisopropylamine dichloroacetate, 4-aminomethyl cyclohexanecarboxylic acid and other active ingredients; tocopherol, carotinoids, flavonoids, tannins, lignans, saponins, butylated hydroxyanisole, dibutylhydroxytoluene, phytic acid and other anti-oxidants, α-hydroxy acids, β-hydroxy acids, and other cell activators; γ-orizanol, vitamin E derivatives, and other circulation-accelerating agents; retinol, retinol derivatives, and other wound healing agents, cepharanthin, cayenne tincture, hinokitiol, iodized garlic extract, pyridoxine hydrochloride, dl-α-tocopherol, dl-α-tocopherol acetate, nicotinic acid, nicotinic acid derivatives, calcium pantothenate, D-pantothenyl alcohol, acetyl pantothenyl ethyl ether, biotin, allantoin, isopropyl methyl phenol, estradiol, ethynyl estradiol, capronium chloride, benzalkonium chloride, diphenhydramine hydrochloride, Takanal (TM), camphor, salicylic acid, nonylic acid vanillylamide, nonanoic acid vanillylamide, Piroctone olamine, glyceryl pentadecanoate, 1-menthol, camphor and other algefacient agents, mononitroguaiacol, resorcin, γ-aminobutyric acid, benzethonium chloride, mexiletine hydrochloride, auxin, female hormones, cantharis tincture, cyclosporine, zinc pyrithione, hydrocortisone, minoxidil, polyoxyethylene sorbitan monostearate, peppermint oil, sasanishiki extract, or other hair growing agents. These agents should be added in amounts sufficient for imparting to the cosmetic a certain biological activity.

Component (m), the pH adjuster, that is normally added to conventional cosmetics and that can be added to the cosmetic of the present invention is exemplified by lactic acid, citric acid, glycolic acid, succinic acid, tartaric acid, dl-malic acid, potassium carbonate, sodium hydrogencarbonate, ammonium hydrogencarbonate, etc.

Component (o), the water, that is normally added to conventional cosmetics and is added to the cosmetic of the present invention is the same type as described earlier.

Component (p), the solvent, that is normally added to conventional cosmetics and that can be added to the cosmetic of the present invention is exemplified by ethers, other than compounds exemplified as component (d); examples of propellants includes LPG, N-methylpyrrolidone, next-generation chlorofluorocarbons, etc.

Component (n), the antioxidant, that is normally added to conventional cosmetics and that can be added to the cosmetic of the present invention is exemplified by tocopherol, butylated hydroxyanisole, dibutylhydroxytoluene, phytic acid, carotenoid, flavonoid, tannin, lignan, or saponin. This agent should be added in an amount sufficient to protect the cosmetic from oxidation.

Component (q), the chelating agent, that is normally added to conventional cosmetics and that can be added to the cosmetic of the present invention is used for making ions of mineral substances insoluble in water. The chelating agent is exemplified by EDTA, alanine, sodium salt of edetic acid, sodium polyphosphate, sodium metaphosphate, or phosphoric acid.

Component (r), the fragrance, that is normally added to conventional cosmetics and that can be added to the cosmetic of the present invention is used for imparting to the cosmetic a certain aroma, scent, or for masking unpleasant odor. There are no special restrictions with regard to the type of fragrance, provided that it is a conventional fragrance normally added to cosmetics, and examples of this component include various extracts shown above as biologically active components; extracts from flowers, seeds, leaves, and roots of various plants; fragrances extracted from seaweeds; fragrances extracted from various parts or secretion glands of animals (e.g., musk and sperm oil), or artificially synthesized fragrances (e.g., menthol, musk, ethyl acetate, or vanilla).
Component (s), the coloring agent, that is normally added to conventional cosmetics and that can be added to the cosmetic of the present invention is exemplified by dyes, pigments, fluorescent bleaches; the dyes are represented by water-soluble dyes, oil-soluble dyes, natural dyes, synthetic dyes, etc.; pigments are represented by extender pigments, inorganic pigments, organic pigments, etc.

There are no special restrictions with regard to the cosmetic, i.e., cosmetic product of the present invention that comprises a mixture of components (a) to (c), or components (a) to (d), or components (a) to (e), provided that the aforementioned compositions improve properties of the cosmetic. In general, the cosmetic of the present invention is exemplified by skin cosmetic products, such as skin cleansing products, skin care products, makeup products, antiperspirant products, and UV-ray protective products, etc.; by hair cosmetic products, such as hair cleansing products, hair styling products, hair dyeing products, hair maintenance products, hair rinse products, etc; by bath cosmetic products; and perfumed water or colognes.

Preferable cosmetic of the present invention is a skin cosmetic, i.e., skin cosmetic product or skin cosmetic composition, or hair cosmetic, i.e., hair cosmetic product or hair cosmetic composition, of which the skin cosmetic is most preferable.

The skin cosmetic, i.e., skin cosmetic product skin or skin cosmetic composition mentioned above can be used in various locations, such as on the scalp, face (including lips, eyebrows, cheeks), fingers, nails, and the entire body. Specifically, such skin cosmetic, i.e., skin cosmetic product is exemplified by cleansing gel, cleansing cream, cleansing foam, cleansing milk, cleansing lotion, facial cleansing cream, eye make-up remover, cleansing foam, liquid whole-body soap, hand soap, gel soap, bar soap, facial rinse, body rinse, shaving cream, nail polish remover, anti-acne product, and other skin cleansing products; skin cream, hot oil treatment for the scalp, skin milk, milk lotion, emulsion, toilet water, moisturizers, beauty liquid, facial compact powder, body powder, essences, shaving lotions and other skin care products; foundation, make-up base, white powder, face powder, lipstick, lip cream, lip color, lip-gloss, eye shadow, eyeliner, eye cream, eyebrow pencil, eyelash cosmetic products, eyebrow pencil, eyebrow brush, mascara, rouge, cheek cosmetic products (cheek color, cheek rouge), nail polish, toe polish, nail color, nail lacquer, enamel remover, and other makeup products; deodorants and other antiperspirants; sunscreen, suntanning drugs (suntanning agents) and other UV light protective products.

The aforementioned hair cosmetic, i.e., hair cosmetic product is exemplified by hair cleansing agents, such as shampoo, shampoo with rinse, etc.; hair oil, hair curl retaining agents, setting agents, hair cream, hair spray, hair liquid, and other hair styling products; hair dyes, hair color spray, hair color rinse, hair color stick, and other hair coloring products; hair tonic, hair treatment, hair packs, and other hair maintenance products; and oil rinse, cream rinse, treatment rinse, and other hair rinse products, and in addition the above-mentioned bath cosmetic products are exemplified by bath oil, bath salts, and foam bath products.

There are no particular limitations concerning the form and properties of the cosmetic of the present invention, but it is preferable that when the water-containing cosmetic of the present invention is prepared as a composition consisting of components (a) to (c) and mixed with water, it should be in the form of an oil-in-water type emulsion, in particular an oil-in-water type emulsion having average emulsion particles not exceeding 10.0 μm, when measured by the laser diffraction/dispersion method. Furthermore, when the water-containing cosmetic of the present invention is prepared by mixing a composition consisting of components (a) to (d) or components (a) to (e) and mixed with water, it should be in the form of an oil-in-water type emulsion, in particular an oil-in-water type emulsion having average emulsion particles less than 0.5 μm (500 nm), and in particular less than 0.2 μm (200 nm), when measured by the laser diffraction/dispersion method.

Since the present composition for the preparation of cosmetics that comprises a mixture of aforementioned components (a) to (c) which are mutually compatible, a mixture of aforementioned components (a) to (d) which are mutually compatible, or a mixture of aforementioned components (a) to (e) which are mutually compatible is easily emulsified by mixing with water, a stable oil-in-water emulsion with small or microscopic particle size can be obtained by mixing the composition with an appropriate amount of water with the use of a simple mixer or stirrer such as paddle-blade stirrer, propeller stirrer, Henschel mixer, TK Homomixer (product of Tokushu Kaka Kogyo Co., Ltd.), TK Homodisper (product of Tokushu Kika Kogyo Co., Ltd.), or similar well known simple mixers.

In other words, a stable oil-in-water type emulsion with particles having dimensions from small to microscopic can be prepared by using a simple stirring, mixing, or emulsifying devices without the use of a high-pressure emulsifier or high shear force emulsifier such as a colloidal mill, or colloidal mixer.
What is meant here under the term “high-pressure emulsifier” is a high pressure device used for crushing emulsified organopolysiloxane particles in a primary emulsion to create uniform and microscopic emulsified particles therein. In this device, the primary emulsion is subjected to high pressure to become high-speed flows, the high-speed flows are branched, and the branched high-speed flows are collided each other, or the high-speed flows are passed through adjustable microscopic gaps, and are collided to impact rings or rotating bodies, and the organopolysiloxane particles in the primary emulsion are crushed by colliding forces, shear forces, cavitation, turbulence, or ultrasonic waves generated in the aforementioned collisions to become uniform microscopic emulsified particles.
Specific examples of such devices include super-high-pressure Gaulin-type homogenizer (the product of Gaulin Co.), high-pressure homogenizer (the product of Izumi Food Machinery Co., Ltd.), high-pressure homogenizer (the product of Rannie Co.), Microfluidizer (the product of Microfluidics Co.), and Nanomizer (the product of Nanomizer Co.).

For preparing the cosmetic, in particular, the oil-in-water type emulsion cosmetic by using the composition for the preparation of cosmetics, a composition for the preparation of cosmetics is manufactured at first by uniformly mixing components (a) to (c), or components (a) to (d), or components (a) to (e) with the use of an arbitrary stirrers or mixer. The obtained composition is then gradually loaded at room temperature into an arbitrary stirrer or mixer while water alone or water that contains arbitrary components is stirred therein at a speed of 500 to 5000 rpm. After the composition is loaded, stirring and mixing should be continued. Although the mass ratio of the composition for the preparation of cosmetics and water may be arbitrary, for improved temporal stability of the obtained oil-in-water type emulsion cosmetic, it is beneficial that the above ratio be in the range of 0.05/0.95 to 0.50/0.50.

When the cosmetic, in particular oil-in-water type emulsion cosmetic having an average particle size less than 0.5 μm, is prepared with the use of the composition for the preparation of cosmetics of the present invention, a composition for the preparation of cosmetics is first prepared by mixing aforementioned components (a) to (c), or components (a) to (d), or components (a) to (e) with the use of an arbitrary stirrer or mixer, the obtained composition is then gradually loaded into an aqueous phase at room temperature an arbitrary stirrer or mixer which is stirred by an arbitrary stirrer or mixer in at a speed of 100 to 5000 rpm. The cosmetic and, in particular the oil-in-water type emulsion cosmetic with an average particle size smaller than 0.5 μm obtained by the above-described method, has an appearance from semi-transparency to white turbidity, and is characterized by excellent temporal stability. Although the mass ratio of the composition for the preparation of cosmetics and water may be arbitrary, for improved temporal stability of the obtained oil-in-water type emulsion cosmetic, it is beneficial that the above ratio be in the range of 0.01/0.99 to 0.50/0.50.

When the production scale is small, an oil-in-water type emulsion cosmetic, such as toilet water or beauty liquid, can be obtained by stirring water in a container manually with the use of a spatula and gradually adding the aforementioned composition for the preparation of cosmetics into the water. Such cosmetic possesses high temporal stability and excellent feel of use, and does not need of detailed investigation of emulsification conditions generally required for obtaining the stable oil-in-water type emulsion cosmetic.

The cosmetic of the present invention and the water-containing cosmetic that has been prepared by using the composition for the preparation of cosmetics of the present invention possess excellent temporal stability, uniform appearance, and excellent feel of use. The water-containing cosmetic of the present invention can be easily prepared by mixing the inventive composition for the preparation of cosmetics with water (used in such an amount that does not cause emulsification). The stirring and mixing devices suitable for this purpose are exemplified by paddle-blade stirrers, propeller-type stirrers, Henschel-type stirrer, and TK Homomixer (of Tokushu Kogyo Co., Ltd.), TK Homodisper (of Tokushu Kogyo Co., Ltd.).

There are no restrictions concerning containers used for the cosmetic, namely, cosmetic product of the present invention. Examples of the container include jars, pump cans, tubes, bottles, pressure spray containers, pressure-resistant aerosol containers, light-resistant containers, compact containers, metal cans, lipstick containers, dispensing containers, aerosol containers, partitioned containers with mixed fluid discharge outlets, etc., and jars, pump, bottles, and pressure spray containers for the oil-in-water type emulsion cosmetic.

Normally, when component (c) is a hydrophobic silicone oil or contains such an oil, an oil phase that is composed of the hydrophobic silicone oil reveals a tendency to separate. Therefore, when a transparent container is filled with such a product, the line of separation of phases becomes clearly visible, and this may spoil the appearance of the product. However, since the oil-in-water type emulsion cosmetic that contains the composition for the preparation of cosmetics of the present invention possesses excellent temporal stability, the oil-in-water type emulsion cosmetic of the present invention can be stored stably even if filled in such transparent container, and this makes the cosmetic as a product with excellent appearance well sellable by stores.

### EXAMPLES

The present invention will be further described in more detail with reference to practical examples and comparative examples, though it is understood that these examples should not be construed as limiting the scope of the present invention. The dimethylsiloxane•methylhydrogensiloxane copolymer and methylhydrogensiloxane as well as the dimethylpolysiloxanes used in synthesis examples were produced by equilibrium polymerization. The following measurement methods were used in the subsequent synthesis examples, practical examples, and comparative examples.

Viscosity of Polyoxyalkylene-Modified Dimethylpolysiloxane

Viscosity of a 50 mass % dipropyleneglycol solution of a polyoxyalkylene-modified dimethylpolysiloxane obtained in Synthesis Examples 1 to 7 was measured by means of a rotary-type viscosimeter of Shibaura System Co., Ltd. (trademark: Vismetron VDA-L type, Rotor No. 3); measurement temperature: 25° C.

Appearance of the Composition for the Preparation of Cosmetics

Appearance of the composition for the preparation of cosmetics was evaluated by visual observation. The following criteria were used.

TABLE 1

| | |
|---|---|
| ⊚ | Completely transparent |
| ◯ | Transparent with some turbidity |
| Δ | Somewhat non-uniform and somewhat turbid |

Test of the O/W type Emulsion Cosmetic for Temporal Stability

The oil-in-water, i.e., O/W type emulsion cosmetic was kept in a quiescent state in a thermostat at room temperature and at 40° C., and then the appearance of the oil-in-water type emulsion cosmetic was observed immediately after preparation and after storage for one month in the thermostat. Criteria for evaluation are shown below.

TABLE 2

| | |
|---|---|
| ⊚ | No separation of oil phase was observed |
| ◯ | Slight separation of oil phase was observed |
| Δ | Separation of oil phase was clearly observed |
| X | Emulsion could not be obtained |

Average Particle Size of Emulsion Particles in O/W Type Emulsion Cosmetic

The average particle size of emulsion particles in the oil-in-water type emulsion cosmetic was measured by means of a submicron-particle analyzer (the product of Coulter Electronics Company; Coulter Model N4) for measuring submicron-size particles by a laser diffraction/dispersion method. Furthermore, the average diameter of emulsion particles contained in the oil-in-water type emulsion cosmetic which contains large emulsion particles with the average particle size of exceeding 1.0 μm when measured by means of a submicron-particle analyzer was determined from the median diameter measured by means of a laser-diffraction-type particle distribution analyzer (the product of Horiba Company; Model LA-500). The oil-in-water type emulsion was considered to be turbid when any uniform oil-in-water type emulsion could not be prepared, the whole emulsion was turbid, and, therefore, any particle size could not be measured.

Feel of Use of Cosmetic

This criterion was evaluated by a panel of 20 women who observed the cosmetic with regard to stickiness, smoothness, and softness of touch etc.

### Synthesis Example 1

A glass flask equipped with a stirrer and a thermometer was loaded with 15.9 parts by weight of a dimethylsiloxane•methylhydrogensiloxane copolymer represented by the following average structural formula (7):

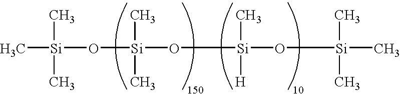

and 34.1 parts by weight of a polyoxyalkylene (oxyethylene and oxypropyrene random copolymer) allyletherified on one of the molecular terminals of the following structural formula (8):

CH₂═CHCH₂O(C₂H₄O)₃₀(C₃H₆O)₁₀H

(with the ratio of silicon-bonded hydrogen atoms of the aforementioned copolymer to allyl groups of the aforementioned allyl-etherified polyoxyalkylene equal to 1:1.3). The components were mixed, and then 15 parts by weight of isopropyl alcohol as a solvent were added. Furthermore, chloroplatinic acid (in an amount of 15 ppm per total weight of the reaction-starting materials) was added, the content was stirred for 2 hours at 86° C., and the obtained product was subjected to infrared spectroscopic analysis that confirmed that peaks corresponding to silicon-bonded hydrogen atoms were absent. Following this, 50 parts by weight of dipropyleneglycol were added, and after stirring for 30 minutes, the product was subjected to stripping in vacuum, whereby a 50 wt. % solution of polyoxyalkylene-modified dimethylpolysiloxane of the average structural formula (9):

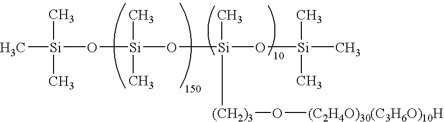

was obtained (viscosity: 4,780 mPa·s). Hereinafter, this product will be referred to as “Silicone Solution No. 1.”

### Synthesis Example 2

A glass flask equipped with a stirrer and a thermometer was loaded with 19 parts by weight of a dimethylsiloxane•methylhydrogensiloxane copolymer represented by the following average structural formula (10):

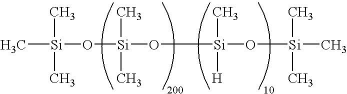

and 31 parts by weight of the same polyoxyalkylene (oxyethylene and oxypropyrene random copolymer) allyletherified on one of the molecular terminals that was used in Synthesis Example 1 (with the ratio of silicon-bonded hydrogen atoms of the aforementioned copolymer to allyl groups of the aforementioned allyl-etherified polyoxyalkylene equal to 1:1.3). The components were mixed, and then 15 parts by weight of isopropyl alcohol as a solvent were added. Furthermore, chloroplatinic acid (in an amount of 15 ppm per total weight of the reaction-starting materials) was added, the content was stirred for 2 hours at 86° C., and the obtained product was subjected to infrared spectroscopic analysis that confirmed that peaks corresponding to silicon-bonded hydrogen atoms were absent. Following this, 50 parts by weight of dipropyleneglycol were added, and after stirring for 30 minutes, the product was subjected to stripping in vacuum, whereby a 50 wt. % solution of polyoxyalkylene-modified dimethylpolysiloxane of average structural formula (11):

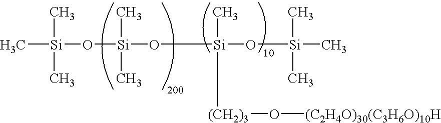

was obtained (viscosity: 24,000 mPa·s). Hereinafter, this product will be referred to as “Silicone Solution No. 2.”

### Synthesis Example 3

A glass flask equipped with a stirrer and a thermometer was loaded with 27.2 parts by weight of a dimethylsiloxane•methylhydrogensiloxane copolymer, represented by the following average structural formula (12):

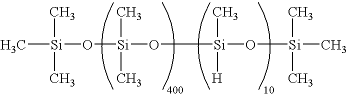

and 22.8 parts by weight of polyoxyalkylene allyletherified on one of the molecular terminals with allylether of the following formula (13):

CH₂C═CHCH₂O(C₂H₄O)₁₉(C₃H₆O)₁₉H

(with the ratio of silicon-bonded hydrogen atoms of the aforementioned copolymer to allyl groups of the aforementioned allyl-etherified polyoxyalkylene equal to 1:1.3). The components were mixed, and then 15 parts by weight of a isopropyl alcohol as a solvent were added. Furthermore, chloroplatinic acid (in an amount of 15 ppm per total weight of the reaction-starting materials) was added, the content was stirred for 2 hours at 86° C., and the obtained product was subjected to infrared spectroscopic analysis that confirmed that peaks corresponding to silicon-bonded hydrogen atoms were absent. Following this, 50 parts by weight of dipropyleneglycol were added, and after stirring for 30 minutes, the product was subjected to stripping in vacuum, whereby a 50 wt. % solution of polyoxyalkylene-modified dimethylpolysiloxane of average structural formula (14):

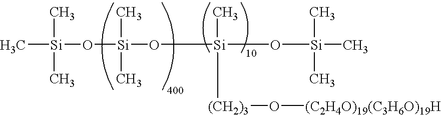

was obtained (viscosity: 30,000 mPa·s). Hereinafter, this product will be referred to as “Silicone Solution No. 3.”

### Synthesis Example 4

A glass flask equipped with a stirrer and a thermometer was loaded with 29.9 parts by weight of dimethylpolysiloxane of the following average structural formula (15):

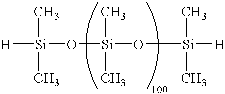

and 20.1 parts by weight of the same polyoxyalkylene allyletherified on one of the molecular terminals that was used in Synthesis Example 1 (with the ratio of silicon-bonded hydrogen atoms of the aforementioned copolymer to allyl groups of the aforementioned allyl-etherified polyoxyalkylene equal to 1:1.3). The components were mixed, and then 15 parts by weight of isopropyl alcohol as a solvent were added. Furthermore, chloroplatinic acid (in an amount of 15 ppm per total weight of the reaction-starting materials) was added, the content was stirred for 2 hours at 86° C., and the obtained product was subjected to infrared spectroscopic analysis that confirmed that peaks corresponding to silicon-bonded hydrogen atoms were absent. Following this, 50 parts by weight of dipropyleneglycol were added, and after stirring for 30 minutes, the product was subjected to stripping in vacuum, whereby a 50 wt. % solution of polyoxyalkylene-modified dimethylpolysiloxane of average structural formula (16):

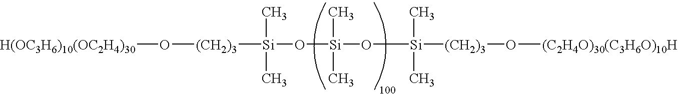

was obtained (viscosity: 30,000 mPa·s). Hereinafter, this product will be referred to as “Silicone Solution No. 4.”

### Synthesis Example 5

In Synthesis Example 1, dipropyleneglycol was not added to the obtained product, and after stripping in vacuum 50 parts by weight of ethanol was added to the stripped product, and the stripped product was stirred for 30 minutes, whereby a 50 wt. % solution of polyoxyalkylene-modified dimethylpolysiloxane of average structural formula (17):

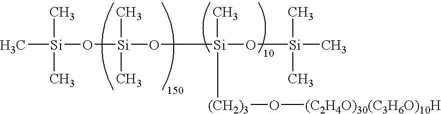

was obtained (viscosity: below 800 mPa·s). Hereinafter, this product will be referred to as “Silicone Solution No. 5.”

### Synthesis Example 6

A glass flask equipped with a stirrer and a thermometer was loaded with 35.1 parts by weight of a dimethylpolysiloxane of the following average structural formula (18):

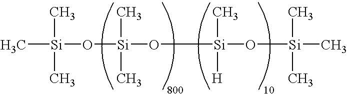

and 14.9 parts by weight of the same polyoxyalkylene allyletherified on one of the molecular terminals that was used in Synthesis Example 3 (with the ratio of silicon-bonded hydrogen atoms of the aforementioned copolymer to allyl groups of the aforementioned allyl-etherified polyoxyalkylene equal to 1:1.3). The components were mixed, and then 15 parts by weight of isopropyl alcohol as a solvent were added. Furthermore, chloroplatinic acid (in an amount of 15 ppm per total weight of the reaction-starting materials) was added, the content was stirred for 2 hours at 86° C., and the obtained product was subjected to infrared spectroscopic analysis that confirmed that peaks corresponding to silicon-bonded hydrogen atoms were absent. Following this, 50 parts by weight of dipropyleneglycol were added, and after stirring for 30 minutes, the product was subjected to stripping in vacuum, whereby a 50 wt. % solution of polyoxyalkylene-modified dimethylpolysiloxane of average structural formula (19):

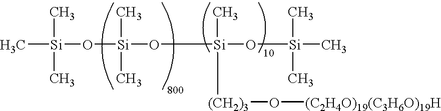

was obtained (viscosity: 100,000 mPa·s). Hereinafter, this product will be referred to as “Silicone Solution No. 6.”

### Synthesis Example 7

A glass flask equipped with a stirrer and a thermometer was loaded with 10.4 parts by weight of a dimethylsiloxane•methylhydrogensiloxane copolymer of the following average structural formula (20):

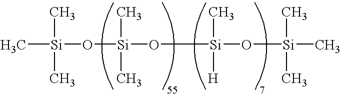

and 39.6 parts by weight of the same polyoxyalkylene allyletherified on one of the molecular terminals that was used in Synthesis Example 1 (with the ratio of silicon-bonded hydrogen atoms of the aforementioned copolymer to allyl groups of the aforementioned allyl-etherified polyoxyalkylene equal to 1:1.3). The components were mixed, and then 30 parts by weight of isopropyl alcohol as a solvent were added. Furthermore, chloroplatinic acid (in an amount of 15 ppm per total weight of the reaction-starting materials) was added, the content was stirred for 2 hours at 86° C., and the obtained product was subjected to infrared spectroscopic analysis that confirmed that peaks corresponding to silicon-bonded hydrogen atoms were absent. Following this, 50 parts by weight of dipropyleneglycol were added, and after stirring for 30 minutes, the product was subjected to stripping in vacuum, whereby a 50 wt. % solution of polyoxyalkylene-modified dimethylpolysiloxane of average structural formula (21):

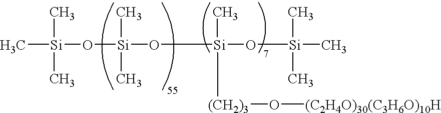

was obtained (viscosity: 500 mPa·s). Hereinafter, this product will be referred to as “Silicone Solution No. 7.”

For comparison, a dimethylpolysiloxane of the average structural formula (22) given below (viscosity: 500 mPa·s) (hereinafter referred to as “Silicone No. 8”) was procured.

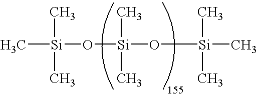

The values of “m”, “n”, “a”, and “b” for respective average structural formulas of the polyoxyalkylene-modified dimethylpolysiloxane of aforementioned Silicone Solution Nos. 1 to 7 and Silicone No. 8 are given in Table 3.

TABLE 3

| Silicone No. | m | n | m + n | m/n | a | b | a + b |
|---|---|---|---|---|---|---|---|
| 1 | 150 | 10 | 160 | 15 | 30 | 10 | 40 |
| 2 | 200 | 10 | 210 | 20 | 30 | 10 | 40 |
| 3 | 400 | 10 | 410 | 40 | 19 | 19 | 38 |
| 4 | 100 | 0 | 100 | — | 30 | 10 | 40 |
| 5 | 150 | 10 | 160 | 15 | 30 | 10 | 40 |
| 6 | 800 | 10 | 810 | 80 | 19 | 19 | 38 |
| 7 | 55 | 7 | 62 | 8 | 30 | 10 | 40 |
| 8 | 155 | 0 | 155 | — | — | — | — |

Practical Examples 1 to 20; Comparative Examples 1 to 5 Components shown in Tables 5 and 6 below were uniformly mixed in a beaker with the use of a Three-One Motor (Model LR500B manufactured by Yamato Scientific Co., Ltd.) operating at 300 rpm, whereby composition for the preparation of cosmetics Nos. 1 to 25 were prepared. Numbers indicated in the column relating to the number of the silicone solution in Tables 5 and 6 correspond to Silicone Solution Nos. 1 to 7 and Silicone Number 8 described in aforementioned Synthesis Examples. Furthermore, “parts” in the above tables are “parts by weight”. Also, component (b), polyoxyalkylene-modified dimethylpolysiloxane¹, dimethylpolysiloxane², and methylphenylpolysiloxane³ which are given in Tables 5 and 6 are as follow;

TABLE 4
Trademarks and Manufacturers of Component (b)

| Names of Components | Trademark or Tradename | Manufacturer |
|---|---|---|
| di-POE (8) alkyl (12 to 15) etherphosphoric | Nikkol Chemicals DDP-8 | Nikko Chemicals Co. |
| acid | | |
| Sodium tri-POE (4) lauryl ether phosphate | Hostarhat KL 340D | Clariant in Japan |
| N-coconut oil fatty acid acyl-L-glutaminic | Amisoft CA | Kao Corporation |
| acid | | |
| Disodium polyoxyethylenealkyl (12 to 14) | Kohakule L-400 | Toho Chemical |
| sulfosuccinate | | Industry, Ltd. |
| Polyoxyethylene sorbitane monooleate | Reodol TW-0120V | Kao Corporation |
| Polyoxyethylene sorbitane monostearate | Reodol Supper TW-S120 | Kao Corporation |
| POE-60 hardened castor oil | Nikkol HCO-60 | Nikko Chemicals Co. |
| Disodium PEG-12 | MACKANATE DC-100 | McIntyre |
| dimethiconesulfosuccinate⁴ | | |

Polyoxyalkylene-modified dimethylpolysiloxane¹; polyoxyalkylene-modified dimethylpolysiloxane of the average structural formula given below (kinematic viscosity: 1,700 mm²/s):

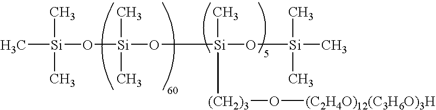

dimethylpolysiloxane²: SH 200C-6cs (the product of Dow Corning Toray Co., Ltd. that comprises dimethylpolysiloxane capped at both molecular terminals with trimethylsiloxy groups; kinematic viscosity: 6 mm²/s), methylphenylpolysiloxane³: SH 556 (the product of Dow Corning Toray Co., Ltd. that comprises phenyltrimethicone; kinematic viscosity: 20 mm²/s), disodium PEG-12 dimethiconesulfosuccinate⁴: disodium salt of sulfosuccinic acid ester of polyoxyethylene-(12)-modified dimethylpolysiloxane.

TABLE 5
Contents of Components and Appearance of Compositions

| | | Practical Example | | | | | | | | | | | | |
|---|---|---|---|---|---|---|---|---|---|---|---|---|---|---|
| | | Nos. of Practical and | | | | | | | | | | | | |
| | | Comparative Example | | | | | | | | | | | | |
| | | 1 | 2 | 3 | 4 | 5 | 6 | 7 | 8 | 9 | 10 | 11 | 12 | 13 |
| | Nos. of composition for the | 1 | 2 | 3 | 4 | 5 | 6 | 7 | 8 | 9 | 10 | 11 | 12 | 13 |
| | preparation of cosmetics | | | | | | | | | | | | | |
| (a) | Silicone Solution No. | 1 | 1 | 2 | 3 | 1 | 1 | 1 | 1 | 1 | 1 | 1 | 4 | 4 |
| Parts | | 6 | 5 | 5 | 10 | 10 | 10 | 5 | 5 | 5 | 5 | 5 | 5 | 5 |
| (b) | Di-POE (8) alkyl (12 to | | 1 | | | | 1 | 1 | 1 | 1 | 1 | 1 | 1 | 1 |
| Parts | 15) etherphosphate | | | | | | | | | | | | | |
| | Sodium tri-POE (4) | 0.5 | | 1 | | | | | | | | | | |
| | lauryletherphosphate | | | | | | | | | | | | | |
| | N-coconut oil fatty acid | | | | 3 | | | | | | | | | |
| | acyl-L-glutaminic acid | | | | | | | | | | | | | |
| | Disodium POE alkyl (12-14) | | | | | 1 | | | | | | | | |
| | sulfosuccinate | | | | | | | | | | | | | |
| | Polyoxyethylene (20) | | | | | | 5 | | | | | | | |
| | sorbitane monolaurate | | | | | | | | | | | | | |
| | POE (60) hardened castor oil | | | | | | | 5 | | | | | | |
| | Disodium PEG-12 | | | | | | | | | | | | | |
| | dimethicone sulfosuccinate⁴ | | | | | | | | | | | | | |
| | Polyoxyalkylene-modified | 4.5 | 5 | 1 | 2 | 7 | | | 5 | 5 | 5 | 5 | 5 | 5 |
| | dimethylpolysiloxane¹ | | | | | | | | | | | | | |
| (c) | Dimethylpolysiloxane² | 43 | 5 | | | | 5 | 5 | | | 5 | 5 | 5 | 10 |
| Parts | Methylphenylpolysiloxane³ | 3 | 5 | | | | 5 | 5 | | | 5 | 5 | 5 | 10 |
| | Decamethylcyclopentasiloxane | 43 | | 3 | 8 | 5 | | | | | | | | |
| | Liquid paraffin | | | | | | | | 5 | | | | | |
| | Squalane | | | | | | | | 5 | | | | | |
| | Isononyl isononate | | | | | | | | | 5 | | | | |
| (d) | Ethanol | — | 79 | 90 | 77 | 77 | 74 | 79 | 79 | 84 | | 40 | 79 | 69 |
| Parts | Isopropanol | | | | | | | | | | 79 | | | |
| | 1,3-butylene glycol | | | | | | | | | | | 39 | | |
| (e) | Water | — | — | — | — | — | — | — | — | — | — | — | — | — |
| Parts | | | | | | | | | | | | | | |

| | | | | | | | | | | | | | |
|---|---|---|---|---|---|---|---|---|---|---|---|---|---|
| Appearance of Composition | Δ | ⊚ | ⊚ | ⊚ | ⊚ | ⊚ | ⊚ | ⊚ | ⊚ | ⊚ | ◯ | ⊚ | ⊚ |

TABLE 6
Contents of Components and Appearance of Compositions

| | | | | | | | | | Comparative | | | | |
|---|---|---|---|---|---|---|---|---|---|---|---|---|---|
| | | Practical Example | | | | | | | Example | | | | |
| | | Nos. of Practical and | | | | | | | | | | | |
| | | Comparative Example | | | | | | | | | | | |
| | | 14 | 15 | 16 | 17 | 18 | 19 | 20 | 1 | 2 | 3 | 4 | 5 |
| | Nos. of composition for the | 14 | 15 | 16 | 17 | 18 | 19 | 20 | 21 | 22 | 23 | 24 | 25 |
| | preparation of cosmetics | | | | | | | | | | | | |
| (a) | Silicone Solution No. | 5 | 1 | 1 | 1 | 1 | 1 | 1 | 6 | 7 | 8 | — | 1 |
| Parts | | 5 | 15 | 18 | 14 | 10 | 10 | 10 | 5 | 5 | 5 | 5 | 5 |
| (b) Parts | di-POE (8) alkyl (12 to 15) | 1 | 5 | 3 | | 1.5 | | | 1 | 1 | 1 | | — |
| | etherphosphate | | | | | | | | | | | | |
| | Sodium tri-POE (4) | | | | | | | | | | | 1 | |
| | lauryletherphosphate | | | | | | | | | | | | |
| | N-coconut oil fatty acid | | | | | | | | | | | | |
| | acyl-L-glutaminic acid | | | | | | | | | | | | |
| | Disodium POE alkyl (12-14) | | | | | | | | | | | | |
| | sulfosuccinate | | | | | | | | | | | | |
| | Polyoxyethylene (20) | | | | | | 1.5 | | | | | 1 | |
| | sorbitane monolaurate | | | | | | | | | | | | |
| | POE (60) hardened castor oil | | | | | | | | | | | | |
| | Disodium PEG-12 dimethicone | | | | 2 | | | | | | | | |
| | sulfosuccinate⁴ | | | | | | | | | | | | |
| | Polyoxyalkylene-modified | 5 | 15 | 13 | 10 | | | 8 | 5 | 5 | 5 | | |
| | dimethylpolysiloxane¹ | | | | | | | | | | | | |
| (c) Parts | Dimethylpolysiloxane² | 5 | 10 | 8 | 6 | 5 | 5 | 5 | 5 | 5 | 5 | | 5 |
| | Methylphenylpolysiloxane³ | 5 | 10 | 8 | 6 | 5 | 5 | 5 | 5 | 5 | 5 | | 5 |
| | Decamethylcyclopentasiloxane | | | | | | | | | | | 3 | |
| (d) Parts | Ethanol | 79 | 50 | 25 | 39 | 78.5 | 78.5 | 72 | 79 | 89 | 79 | 95 | 85 |
| (e) Parts | Water | — | — | 25 | 23 | — | — | — | — | — | — | — | |

| | | | | | | | | | | | | |
|---|---|---|---|---|---|---|---|---|---|---|---|---|
| Appearance of Composition | ⊚ | ⊚ | ◯ | ◯ | ⊚ | ⊚ | ⊚ | ⊚ | ⊚ | ⊚ | ⊚ | ⊚ |

### Practical Examples 21 to 40; Comparative Examples 6 to 10

Oil-in-water type emulsions were prepared by the method described below based on the use of composition for the preparation of cosmetics Nos. 1 to 20 and composition for the preparation of cosmetics Nos. 21 to 25.

### [Preparation of 0/W Emulsion No. 1]

90 parts by weight of ion-exchanged water were poured into a beaker and, while the water was stirred with the use of a Three-One Motor (Model LR500B manufactured by Yamato Scientific Co., Ltd.) operating at 500 rpm, 10 parts by weight of the composition for the preparation of cosmetics were gradually added, whereby an oil-in-water type emulsion was obtained.

Stability of each obtained oil-in-water type emulsion was evaluated by testing the product with regard to temporal stability by the same method as described above. The average size of emulsion particles was also measured by the previously described method. Results of measurements and evaluations are shown in Tables 7 and 8.

TABLE 7
Properties of O/W type Emulsion

| | Nos. of Practical and | | | | | | | | | | | | |
|---|---|---|---|---|---|---|---|---|---|---|---|---|---|
| | Comparative Examples | | | | | | | | | | | | |
| | 21 | 22 | 23 | 24 | 25 | 26 | 27 | 28 | 29 | 30 | 31 | 32 | 33 |
| Numbers of composition | 1 | 2 | 3 | 4 | 5 | 6 | 7 | 8 | 9 | 10 | 11 | 12 | 13 |
| for the preparation of | | | | | | | | | | | | | |
| cosmetics | | | | | | | | | | | | | |
| Average particle diameter | 4000 | 60 | 58 | 67 | 64 | 85 | 70 | 120 | 85 | 81 | 150 | 66 | 81 |
| (nm) (immediately after | | | | | | | | | | | | | |
| the preparation) | | | | | | | | | | | | | |
| Emulsification conditions | ⊚ | ⊚ | ⊚ | ⊚ | ⊚ | ⊚ | ⊚ | ⊚ | ⊚ | ⊚ | ⊚ | ⊚ | ⊚ |
| immediately after the | | | | | | | | | | | | | |
| Preparation | | | | | | | | | | | | | |
| Temporal stability (after 1 | ⊚ | ⊚ | ⊚ | ⊚ | ⊚ | ⊚ | ⊚ | ⊚ | ⊚ | ⊚ | ◯ | ⊚ | ⊚ |
| months at 40° C.) | | | | | | | | | | | | | |

TABLE 8
Properties of O/W type Emulsion

| | Practical Examples | | | | | | | Comparative Examples | | | | |
|---|---|---|---|---|---|---|---|---|---|---|---|---|
| | Nos. of Practical and | | | | | | | | | | | |
| | Comparative Examples | | | | | | | | | | | |
| | 34 | 35 | 36 | 37 | 38 | 39 | 40 | 6 | 7 | 8 | 9 | 10 |
| Numbers of composition | 14 | 15 | 16 | 17 | 18 | 19 | 20 | 21 | 22 | 23 | 24 | 25 |
| for the preparation of | | | | | | | | | | | | |
| cosmetics | | | | | | | | | | | | |
| Average particle | 68 | 80 | 34 | 55 | 105 | 88 | 56 | Turbid | 89 | Tur-bidi | 110 | 80 |
| diameter (nm) | | | | | | | | | | | | |
| (immediately after the | | | | | | | | | | | | |
| preparation) | | | | | | | | | | | | |
| Emulsification | ⊚ | ⊚ | ⊚ | ⊚ | ⊚ | ⊚ | ⊚ | Δ | ⊚ | X | ⊚ | ⊚ |
| conditions immediately | | | | | | | | | | | | |
| after the Preparation | | | | | | | | | | | | |
| Temporal stability (after | ⊚ | ◯ | ◯ | ◯ | ◯ | ⊚ | ⊚ | Δ | Δ | X | Δ | Δ |
| 1 months at 40° C.) | | | | | | | | | | | | |

### Practical Examples 41 to 43; Comparative Examples 11 to 13

Oil-in-water type emulsion cosmetics were prepared by the method described below based on the use of composition for the preparation of cosmetics Nos. 2 to 4 and composition for the preparation of cosmetics Nos. 21, 24, and 25.

[Preparation of 0/W type Emulsion Cosmetic No. 2]

90 parts by weight of ion-exchanged water were poured into a beaker and, while the water was stirred with the use of a Three-One Motor (Model LR500B manufactured by Yamato Scientific Co., Ltd.) operating at 500 rpm, 9.9 parts by weight of the composition for the preparation of cosmetics and 0.1 parts by weight of liquid paraffin were gradually added, whereby an oil-in-water type emulsion cosmetic was obtained.

Stability of each obtained oil-in-water type emulsion cosmetic was evaluated by testing the product with regard to temporal stability by the same method as described above. The average size of emulsion particles was also measured by the previously described method. Results of measurements and evaluations are shown in Table 9.

TABLE 9
Characteristics of O/W type Emulsion Cosmetics

| | Practical | | | Comparative | | |
|---|---|---|---|---|---|---|
| | Examples | | | Examples | | |
| | Number of Practical and | | | | | |
| | Comparative Examples | | | | | |
| | 41 | 42 | 43 | 11 | 12 | 13 |
| Composition for the | 2 | 3 | 4 | 21 | 24 | 25 |
| preparation of cosmetics | | | | | | |
| Average Particle Size (nm) | 65 | 72 | 80 | Turbid | Turbid | 90 |
| (immediately after preparation) | | | | | | |
| Condition of the emulsion | ⊚ | ⊚ | ⊚ | Δ | Δ | Δ |
| immediately after preparation | | | | | | |
| Temporal Stability | ⊚ | ⊚ | ⊚ | Δ | Δ | Δ |
| (after 1 month at 40° C.) | | | | | | |

As can be seen from Tables 7, 8, and 9, the use of composition for the preparation of cosmetics Nos. 1 to 20 makes it possible to obtain oil-in-water type emulsions of excellent temporal stability (Practical Examples 21 to 43). In particular, when component (d) such as ethanol or the like was used as a dispersing medium (Practical Examples 2 to 20) for the composition for the preparation of cosmetics, the oil-in-water type emulsion with the average particle size of 200 nm or lower and even better temporal stability was obtained.

On the other hand, the use of composition for the preparation of cosmetics Nos. 21 to 25 produced oil-in-water type emulsions which may exhibit turbidity and non-uniformity. Even if it can produce oil-in-water type emulsions exhibiting uniformity immediately after the preparation, the emulsions separated an oil phase temporally after the preparation. In other words, the last-mentioned emulsions, namely, had low stability (see Comparative Examples 6 to 13).

Cosmetics were produced from the aforementioned composition for the preparation of cosmetics and cosmetic raw materials and evaluated by the methods described above. The average particle sizes of the oil-in-water type emulsions were also measured by the above-described methods.

### Practical Example 44

### Preparation of Milky Lotion

| No. | Components | wt. % |
|---|---|---|
| 1 | Composition for the preparation of | 6.9 |
| | cosmetics No. 2 | |
| 2 | Ethanol | 2.0 |
| 3 | Sodium hydrogen phosphate | 0.01 |
| 4 | Sodium dihydrogen phosphate | 0.02 |
| 5 | Paraben | 0.2 |
| 6 | Purified water | Balance |

### (Method of Preparation)

A. Mixing components No. 1, No. 2, and No. 5.
B. Dissolving components No. 3, and No. 4 in No. 6.
C. Gradually adding the mixture of item “A” while stirring the mixture of item “B” at 500 rpm, thus obtaining a milky lotion with emulsion particles having the average diameter of 58 nm.
Evaluation of the obtained milky lotion with regard to the feel of use gave positive results confirming excellent smoothness in combination with feel of moist.

### Practical Example 45

### Preparation of Moisturizing Beauty Liquid

| No. | Components | wt. % |
|---|---|---|
| 1 | Composition for the preparation of | 6.9 |
| | cosmetics No. 2 | |
| 2 | Glycerol | 10.0 |
| 3 | Methylisothiazolynone | 0.05 |
| 4 | Purified water | Balance |
| 5 | Tremella fuciformis Berkeley | 0.3 |
| | Polysaccharide | |
| 6 | Purified water | 29.7 |

### (Method of Preparation)

A. Mixing components No. 2, No. 3, and No. 4.
B. Adding components No. 5 and No. 6 and dissolving the components while heating.
C. Gradually adding component No. 1 to the mixture of item “A” while stirring the latter at 500 rpm.
D. Adding the mixture of item “B” to the mixture of item “C”, mixing both components, thus obtaining a moisturizing beauty liquid with emulsion particles having the average diameter of 64 nm.
Evaluation of the obtained moisturizing beauty liquid with regard to the feel of use gave positive results confirming ease of spreading in combination with fresh sense of use.

### Practical Example 46

### Preparation of Wiping-Off Beauty Liquid

| No. | Components | wt. % |
|---|---|---|
| 1 | Composition for the preparation of cosmetics No. 2 | 20.7 |
| 2 | PEG3 cocoamide | 0.2 |
| 3 | PEG6 cocoamide | 0.5 |
| 4 | Ethanol | 5.0 |
| 5 | Purified water | Balance |
| 6 | Carnosine | 0.1 |
| 7 | Polyoxypropylene methyl glycoside | 0.4 |
| 8 | Carboxyvinyl polymer (0.2% aqueous solution | 1.8 |
| 9 | Sodium hydroxide (1% aqueous solution) | 10 |
| 10 | Citric acid | 0.05 |

### (Method of Preparation)

A. Mixing components Nos. 1 to 4.
B. Dissolving component No. 6 in component No. 5.
C. Gradually adding the mixture of item “A” to the solution of item “B” while stirring the latter at 500 rpm.
D. Sequentially adding components No. 7, 8, 9, and 10 to item “C” and mixing the contents, thus obtaining a semi-transparent wipe-off beauty liquid (oil-in-water type emulsion).
For evaluating the sense of use, each panelist tested the obtained liquid by washing her face. The test showed that the liquid removed the contaminants that could not be removed with the use of a facial foam and produced a feeling of smoothness by touch.

### Practical Example 47

### Preparation of Water-Containing Gel-Like Pack (Wipe-Off Type)

| No. | Components | wt. % |
|---|---|---|
| 1 | Composition for the preparation of cosmetics No. 2 | 6.9 |
| | (Practical Example 2) | |
| 2 | Dimethylpolysiloxane⁵ | 10 |
| 3 | Carboxyvinyl polymer (0.2% aqueous solution) | 22 |
| 4 | Purified water | Balance |
| 5 | Sodium hydroxide (1% aqueous solution) | 12 |
| 6 | Polyoxypropylene methyl glycoside | 5 |
| 7 | 1,3-butyleneglycol | 7 |
| 8 | Ethanol | 5 |
| 9 | Glycerol | 8 |
| 10 | Methylisothazolinone | 0.05 |

Dimethylpolysiloxane⁵: SH200-100 cs (the product of Dow Corning Toray Co., Ltd.)

### (Method of Preparation)

A. Mixing components Nos. 1 and 2.
B. Dissolving component No. 3 in component No. 4.
C. Gradually adding the mixture of item “A” to the solution of item “B” while stirring the latter at 1000 rpm.
D. Adding component No. 5 to item “C” and mixing, then adding components Nos. 6 to 10 and mixing the contents, thus obtaining a semi-transparent gel-type pack.
Evaluation of the obtained gel-type pack with regard to the sense of use showed that it produced moist feeling without stickiness and with a pleasant feel of touch.

### Practical Example 48

### Preparation of Gel-Like Sterilization Cleanser

| No. | Components | wt. % |
|---|---|---|
| 1 | Composition for the preparation of cosmetics No. 2 | 6.9 |
| 2 | Benzalkonium chloride | 0.3 |
| 3 | Ethanol | 70 |
| 4 | Carboxyvinyl polymer | 0.5 |
| 5 | Glycerol | 2.0 |
| 6 | Purified water | Balance |

### (Method of Preparation)

### A. Mixing Components Nos. 1 to 3.

B. Dissolving component No. 4 in component No. 6 while heating.
C. Adding the mixture of item “A” to the solution of item “B” and then adding component No. 5, whereby a semi-transparent water-containing gel-like sterilization cleanser was obtained.
Evaluation of the obtained water-containing gel-like sterilization cleanser with regard to the feel of use showed that the cleanser was characterized by smoothness of touch and had a pleasant feel of touch without feel of stretching.

### INDUSTRIAL APPLICABILITY

The composition for the preparation of cosmetics of the present invention is a cosmetic raw material, i.e., raw material for cosmetics, cosmetic products or cosmetic compositions, or ingredient to be contained in various cosmetics, cosmetic products or cosmetic compositions. The composition for the preparation of cosmetics of the present invention is useful for the production of cosmetics, especially for the production of oil-in-water type emulsion cosmetics and water-containing cosmetics having excellent temporal stability.

The cosmetic of the present invention is useful for caring, protecting and beautifying the human skin, and protecting, and beautifying the human hair.
The inventive method for the production of water-containing cosmetics is useful for producing water-containing cosmetics simply.

1. A composition for the preparation of cosmetics comprising a mixture of
(a) a polyoxyalkylene-modified diorganopolysiloxane represented by the following average structural formula (1): 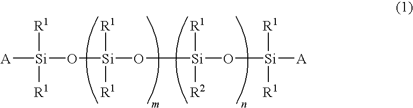
{where R¹ is a monovalent hydrocarbon group or substituted monovalent hydrocarbon group (except for groups corresponding to R²); R² is a polyoxyalkylene group of the following general formula (2): —R³—O—(C₂H₄O)ₐ (C₃H₆O)_{b}R⁴ where R³ is an alkylene group with 2 to 30 carbon atoms; R⁴ is a group selected from hydrogen atoms, an alkyl group with 1 to 30 carbon atoms, or an organic group of the following formula: —(OC)—R⁵ (where R⁵ is an alkyl group with 1 to 30 carbon atoms), “a” and “b” are numbers that satisfy the following conditions: 1≦a≦50; 0≦b≦50; and 10≦(a+b)≦100; and “A” may be the same or different and is selected from a hydroxyl group, R¹ and R²; “m” and “n” satisfy the following conditions: 100≦m≦500; 0≦n=40; but when n=0, at least one “A” is R²;
(b) a surfactant of one or more types (except for surfactants corresponding to component (a)); and
(c) an oil of one or more types.

2. The composition for the preparation of cosmetics according to claim 1, wherein component (a) is present in an amount of 0.85 to 680 parts by mass and component (b) is present in an amount of 1.4 to 1120 parts by mass per 100 parts by mass of component (c).

3. The composition for the preparation of cosmetics according to claim 2, wherein component (a) is present in an amount of 1.0 to 340 parts by mass and component (b) is present in an amount of 1.5 to 560 parts by mass per 100 parts by mass of component (c).

4. The composition for the preparation of cosmetics according to claim 1, further comprising (d) a biologically allowable hydrophilic medium of one or more types.

5. The composition for the preparation of cosmetics according to claim 4, wherein component (d) is present in an amount of 20.0 to 98.4% of the total mass of the composition.

6. The composition for the preparation of cosmetics according to claim 5, wherein component (d) is present in an amount of 40.0 to 98.4% of the total mass of the composition.

7. The composition for the preparation of cosmetics according to claim 5, wherein component (a) is present in an amount of 0.9 to 90 parts by mass, component (b) is present in an amount of 0.6 to 120 parts by mass, and component (c) is present in an amount of 1.1 to 150 parts by mass per 100 parts by mass of component (d).

8. The composition for the preparation of cosmetics according to claim 7, wherein component (a) is present in the amount of 4.0 to 300 parts by mass, and component (b) is present in an amount of 5 to 480 parts by mass per 100 parts by mass of component (c).

9. The composition for the preparation of cosmetics according to claim 1, further comprising (e) water.

10. The composition for the preparation of cosmetics according to claim 9 wherein component (a) is present in an amount of 0.9 to 90 parts by mass, component (b) is present in an amount of 0.6 to 120 parts by mass, and component (c) is present in an amount of 1.1 to 150 parts by mass per 100 parts by mass of component (d), and wherein component (d) is present in an amount of 20.0 to 98.4% of the total mass of the composition, and component (e) is present in an amount not exceeding the content of component (d) and not exceeding 40.0% of the total mass of the composition;

11. The composition for the preparation of cosmetics according to claim 1, wherein the following condition is observed in the average structural formula (1) of component (a): 5≦(m/n)≦50.

12. The composition for the preparation of cosmetics according to claim 1, wherein the viscosity of a 50 wt. % dipropyleneglycol solution of component (a) at 25° C. ranges from 1,000 mPa·s to 60,000 mPa·s.

13. The composition for the preparation of cosmetics according to claim 1, wherein component (b) is composed of at least one (b1) an ionic surfactant and at least one (b2) a non-ionic surfactant.

14. The composition for the preparation of cosmetics according to claim 13, wherein component (b1) is an anionic surfactant or a phospholipid.

15. The composition for the preparation of cosmetics according to claim 14, wherein the anionic surfactant is selected from the group consisting of a polyoxyalkylene alkyl ether phosphoric acid, alkali-metal salt thereof, N-fatty acid acylamino acid, alkali-metal salt of polyoxyalkylene sulfosuccinic acid, and alkali-metal salt of sulfosuccinic acid ester of a polyoxyalkylene-modified dimethylpolysiloxane.

16. The composition for the preparation of cosmetics according to claim 13, wherein component (b2) is selected from the group consisting of a polyoxyalkylene-modified diorganopolytsiloxane (wherein the degree of polymerization in the diorganopolysiloxane portion is lower than that of the portion of component (a), and HLB is in the range of 3 to 10), polyoxyalkylene alkyl ether, sorbitane fatty acid ester, polyoxyalkylenesorbitane fatty acid ester, polyoxyethylene hardened castor oil, and polyoxyalkylene fatty acid ester.

17. The composition for the preparation of cosmetics according to claim 16, wherein the polyorganoalkylene-modified diorganopolysiloxane of component (b2) is represented by the following average structural formula (3): 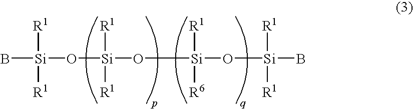
where R¹ is a monovalent hydrocarbon group or substituted monovalent hydrocarbon group (except for groups corresponding to R⁶); R⁶ is a polyoxyalkylene group of the following general formula (4): —R⁷—O—(C₂H₄O)_{d}(C₃H₆O)ₑR⁸ where R⁷ is an alkylene group with 2 to 8 carbon atoms; R⁸ is a group selected from a hydrogen atom, alkyl group with 1 to 12 carbon atoms, and organic group of the following formula: —(OC)—R⁹ (where R⁹ is an alkyl group with 1 to 12 carbon atoms), “d” and “e” are numbers that satisfy the following conditions: 1≦d≦20; 0≦e≦20; and 5≦(d+e)≦40; groups designated by “B” may be the same or different and are selected from a hydroxyl group, R¹ and R⁶; “p” and “q” satisfy the following conditions: 0≦p≦90; 0≦q≦10; but when q=0, at least one “B” is R⁶}.

18. The composition for the preparation of cosmetics according to claim 1, wherein component (c) is a higher fatty acid alkyl ester, hydrocarbon oil, or hydrophobic silicone oil having viscosity of 0.65 mPa·s to 100,000 mPa·s at 25° C.

19. The composition for the preparation of cosmetics according to claim 4, wherein component (d) is a hydrophilic alcohol that has in one molecule at least one hydroxyl group and that is liquid at room temperature.

20. The composition for the preparation of cosmetics according to claim 19, wherein the hydrophilic alcohol is a monovalent or polyvalent alcohol having 2 to 10 carbon atoms.

21. The composition for the preparation of cosmetics according to claim 20, wherein the monovalent or polyvalent alcohol having 2 to 10 carbon atoms is selected from the group consisting of ethanol, isopropyl alcohol, and dipropyleneglycol.

22. The composition for the preparation of cosmetics according to claim 1, wherein the composition for the preparation of cosmetics is a composition for the preparation of oil-in-water type emulsion cosmetics.

23. The composition for the preparation of cosmetics according to claim 1, wherein the composition for the preparation of cosmetic is a composition for the preparation of oil-in-water type emulsion cosmetics with an average particle size, measured by the laser diffraction/dispersion method for emulsion particles, of less than 10.0 μm (10,000 nm).

24. The composition for the preparation of cosmetics according to claim 4, wherein the composition for the preparation of cosmetics is a composition for the preparation of oil-in-water type emulsion cosmetics with an average particle size, measured by a laser diffraction/dispersion method for emulsion particles, of less than 0.20 μm (200 nm).

25. The composition for the preparation of cosmetics according to claim 4, wherein the composition for the preparation of cosmetics is a composition for the preparation of oil-in-water type emulsion cosmetics characterized by the fact that component (d) is present in the amount of 55.0 to 98.4% of the total mass of the composition, and component (a) is present in the amount of 0.9 to 30 parts by mass, component (b) is present in the amount of 0.6 to 40 parts by mass, and component (c) is present in the amount of 1.1 to 50 parts by mass per 100 parts by mass of aforementioned component (d);
component (d) is ethanol alone or a mixture of ethanol with a biologically allowable hydrophilic medium other than ethanol (however, the mass ratio of the aforementioned components in the mixture is not less than 6/4);
component (b) consists of (b1) an ionic surfactant of one or more types and (b2) a nonionic surfactant of one or more types; and
component (c) is a hydrophobic silicone oil having a viscosity of 0.65 mPa·s to 100,000 mPa·s at 25° C., and
an average particle size, measured by a laser diffraction/dispersion method for emulsion particles, is less than 0.10 μm (100 nm).

26. The composition for the preparation of cosmetics according to claim 1, wherein the composition for the preparation of cosmetics is a composition for the preparation of skin cosmetics.

27. A cosmetic that contains the composition for the preparation of cosmetics described in claim 1.

28. An oil-in-water type emulsion cosmetic that contains the composition for the preparation of cosmetics described in claim 22.

29. The cosmetic according to claim 27, wherein the cosmetic is a skin cosmetic.

30. (canceled)
